(19) United States
(12) Patent Application Publication
(10) Pub. No.: US 2021/0128307 A1
(43) Pub. Date: May 6, 2021

(54) OSTEOCHONDRAL/SUBCHONDRAL TREATMENT SYSTEM

(71) Applicant: In2Bones USA, LLC, Memphis, TN (US)

(72) Inventors: Alan G. Taylor, Memphis, TN (US); Rebecca Hawkins Wahl, Escondido, CA (US)

(73) Assignee: In2Bones USA, LLC, Memphis, TN (US)

(21) Appl. No.: 17/064,483

(22) Filed: Oct. 6, 2020

Related U.S. Application Data
(63) Continuation-in-part of application No. 16/718,047, filed on Dec. 17, 2019.
(60) Provisional application No. 62/928,907, filed on Oct. 31, 2019.

(51) Int. Cl. A61F 2/30; A61F 2/46

(52) U.S. Cl. CPC A61F 2/30756; A61F 2/4618; A61F 2002/4658; A61F 2310/00059; A61F 2310/00029; A61F 2310/00371; A61F 2310/00329; A61F 2310/00023

(57) ABSTRACT

An apparatus and methods are provided for a tapered implant for treating osteochondral/subchondral defects. The tapered implant comprises a top portion that includes a shape that approximates an osteochondral/subchondral surface to be replaced. A bottom portion of the tapered implant is configured to be implanted into a hole drilled in bone. A cylindrical sidewall of the tapered implant has a diameter that generally decreases from a first diameter of the top portion to a second diameter of the bottom portion. The tapered implant comprises any homogenous synthetic or natural material suitable for implantation into bone, including any of collagen, human allograft or autograft, animal xenograft, silicone, bioglass, peek, polyethylene, titanium, and cobalt chrome, and any combination thereof.

### PRIORITY

This application is a continuation-in-part of, and claims the benefit of, U.S. patent application, entitled “Osteochondral/Subchondral Treatment System,” filed on Dec. 17, 2019 and having application Ser. No. 16/718,047, which claims the benefit of and priority to U.S. Provisional application, entitled “Osteochondral/Subchondral Treatment System,” filed on Oct. 31, 2019 and having application Ser. No. 62/928,907, which is a continuation-in-part of, and claims the benefit of, U.S. patent application, entitled “Tapered Osteochondral Implant,” filed on May 25, 2018, and having application Ser. No. 15/989,975, the entirety of each of said applications being incorporated herein by reference. This application also claims the benefit of, U.S. patent application, entitled “Engineered Sterile Cartilage Allograft Implant Plug With Sterile, Specific Instrument Kit(s),” filed on Feb. 19, 2016, and having application Ser. No. 15/048,518, which claims the benefit of, and priority to, U.S. Provisional application filed on Feb. 27, 2015 and having application Ser. No. 62/126,053, the entirety of each of said applications being incorporated herein by reference.

### FIELD

Embodiments of the present disclosure generally relate to the field of surgical implants. More specifically, embodiments of the disclosure relate to an apparatus and methods for a tapered implant for treating osteochondral and subchondral defects.

### BACKGROUND

Articular cartilage is a smooth, white tissue which covers the ends of bones where they come together to form joints in humans and many animals so as to facilitate articulation of the joints and protect and cushion the bones. Subchondral bone is the bone that is underneath the cartilage and provides support to the cartilage. Cartilage or subchondral bone may become damaged, however, due to disease, abrupt trauma or prolonged wear. A number of surgical techniques have been developed to treat damaged osteochondral and subchondral defects. Treating osteochondral/subchondral defects is known to relieve pain and facilitate better joint function, as well as potentially delaying or preventing an onset of arthritis. One surgical technique comprises transplantation of a healthy osteochondral graft so as to replace damaged cartilage and encourage new cartilage growth.

Subchondral or osteochondral grafting typically involves removing cartilage and bone tissue of a defect site by coring or reaming to create a cylindrical bore. A tissue scaffold such as a cylindrical cartilage and subchondral bone plug graft is harvested and then implanted into the bore of the prepared defect site. Healing of the graft bone to host bone results in fixation of the plug graft to the surrounding host region.

The plug graft may be an autograft taken from another body region of less strain, such as the hip, skull, or ribs, or the plug graft may be an allograft, harvested from bone taken from other people, that is frozen and stored in a tissue bank. In some instances, the plug graft may be a xenograft that is harvested from animals of a different species. Moreover, many grafting procedures utilize a variety of natural and synthetic tissue scaffolds, with or instead of bone, such as collagen, silicone, acrylics, hydroxyapatite, calcium sulfate, ceramics, and the like, which may be press-fit into the osteochondral or subchondral hole at a patient's defect area. As such, there is an ongoing need for the development of osteochondral grafting capabilities such as that found in, for example, treating damage to articular cartilage in joints. Provided herein are embodiments and methods for a tapered monophasic implant for treating osteochondral defects. Monophasic refers to a uniform material throughout which can include one or more materials manufactured into a single homogenous material.

### SUMMARY

An apparatus and methods are provided for a tapered implant for treating osteochondral/subchondral defects. The tapered implant comprises a top portion that includes a shape that approximates an osteochondral/subchondral surface to be replaced. A bottom portion of the tapered implant is configured to be implanted into a hole drilled in bone. A cylindrical sidewall of the tapered implant has a diameter that generally decreases from a first diameter of the top portion to a second diameter of the bottom portion. The tapered implant comprises any homogenous synthetic or natural material suitable for implantation into bone, including any of collagen, animal xenograft, human allograft, human autograft, silicone, bioglass, peek, polyethylene, titanium, and cobalt chrome, and any combination thereof. In some embodiments, one or more tapered implants are included in a sterile implant system for repairing osteochondral/subchondral defects in various bone joint locations in a patient's body. The sterile implant system includes instruments that are configured for implanting the one or more tapered implants into the patient's body, such that the implant is flush, subflush, or slightly proud of a surrounding native cartilage surface. The instruments may include any one or more of a cartilage punch, a cannulated obturator, a guidewire, a cannulated reamer, an insertion tamp, and a size gauge, as described herein.

In an exemplary embodiment, an implant for treating osteochondral/subchondral defects comprises: a cylindrical member comprised of a monophasic material; a top portion comprising a first diameter; a bottom portion comprising a second diameter; and a tapered sidewall portion disposed between the top portion and the bottom portion.

In another exemplary embodiment, the tapered sidewall portion includes a diameter that decreases from the first diameter to the second diameter. In another exemplary embodiment, the tapered sidewall portion comprises a degree of tapering that is configured to prevent the implant from subsiding into the hole drilled in bone. In another exemplary embodiment, the implant includes a surface area ranging between substantially 0.09 square inches and substantially 3 square inches. In another exemplary embodiment, the first diameter and the second diameter are selected according to a location in a patient that is to be treated. In another exemplary embodiment, a height of the cylindrical member is substantially 10 millimeters (mm), and the first diameter ranges between substantially 5 mm and substantially 10 mm.

In another exemplary embodiment, the top portion includes a shape configured to approximate an osteochondral/subchondral surface to be replaced. In another exemplary embodiment, the shape includes a curvature of the top portion that approximates the curvature of the osteochondral/subchondral surface to be replaced. In another exemplary embodiment, the curvature is either convex, substantially flat, or concave so as to match the anatomy of the osteochondral/subchondral surface.

In another exemplary embodiment, the implant further comprises a rounded periphery that joins the tapered sidewall portion and the bottom portion, the rounded periphery providing a smooth transition surface between the tapered sidewall portion and the bottom portion. In another exemplary embodiment, the implant further comprises a cylindrical sidewall portion disposed between the top portion and the tapered sidewall portion, the cylindrical sidewall portion including a taper half-angle that is less than the taper half-angle of the tapered sidewall portion.

In an exemplary embodiment, a sterile implant system for repairing osteochondral/subchondral defects comprises: one or more tapered implants configured to treat osteochondral/subchondral defects in various bone joint locations in a patient's body, the one or more tapered implants each comprising monophasic material; a multiplicity of instruments including any one or more of size gauge, a punch, an obturator, a guidewire, a cannulated reamer, and an insertion tamp, the multiplicity of instruments being configured for implanting the one or more tapered implants into the patient's body such that the implant is flush, subflush, or slightly proud of a surrounding native cartilage surface; and a size gauge configured to correspond to sizes of the one or more tapered implants and including a central hole configured to receive the guidewire.

In another exemplary embodiment, the one or more tapered implants and the multiplicity of instruments are packaged together in an exterior container suitable for delivery to a practitioner. In another exemplary embodiment, the one or more tapered implants are stored in a first sterile container. In another exemplary embodiment, any one or more of the punch, the obturator, the guidewire, the cannulated reamer, and the insertion tamp are stored in a second sterile container. In another exemplary embodiment, the size gauge is stored in a third sterile container.

In an exemplary embodiment, a method for a sterile implant system for repairing osteochondral/subchondral comprises: configuring one or more tapered implants to treat osteochondral/subchondral defects in various bone joint locations in a patient's body; and combining the one or more tapered implants with a multiplicity of instruments configured for implantation of the one or more tapered implants into the patient's body, the multiplicity of instruments including at least a guidewire, a cannulated reamer, a punch, an insertion tamp, and a size gauge.

In another exemplary embodiment, configuring comprises forming the one or more tapered implants of a homogenous synthetic material, a homogenous natural material, or a combination thereof. In another exemplary embodiment, configuring comprising forming the one or more tapered implants of any one or more of collagen, silicone, bioglass, peek, polyethylene, titanium, and cobalt chrome. In another exemplary embodiment, configuring comprises forming the one or more tapered implants such that the diameters of a top portion of the one or more tapered implants range from substantially 5 mm to substantially 10 mm. In another exemplary embodiment, combining further comprises: storing the one or more tapered implants in a first sterile container; storing any one or more of the multiplicity of instruments in a second sterile container; and storing the size gauge in a third sterile container.

In an exemplary embodiment, an osteochondral/subchondral treatment system comprises: one or more grafts configured to treat an osteochondral/subchondral defect; a sterile instrument kit comprising a multiplicity of instruments including any one or more of size gauge, a punch, an obturator, a guidewire, a reamer, a cannulated reamer, a graft inserter, and an insertion tamp, the multiplicity of instruments being configured for implanting the one or more grafts into a patient's body such that the graft is flush, subflush, or slightly proud of a surrounding native cartilage surface; and a size gauge configured to correspond to sizes of the one or more grafts.

In another exemplary embodiment, the one or more grafts each comprises a cartilage layer coupled with a bone portion suitable for treating the osteochondral/subchondral defect. In another exemplary embodiment, the cartilage layer is comprised of a material that closely matches existing cartilage at an implant location. In another exemplary embodiment, the cartilage layer is comprised of a synthetic implantable material.

In another exemplary embodiment, any one of the one or more grafts is a xenograft that is suitable for being grafted into the patient's body. In another exemplary embodiment, any one of the one or more grafts is an allograft that includes a cartilage layer having a thickness that substantially matches the thickness of existing cartilage at an implant location. In another exemplary embodiment, the one or more grafts include diameters and lengths that depend upon the particular bone joints into which the one or more grafts are to be implanted, the diameters and lengths being configured to correlate with one another and ranging from relatively small to relatively large.

In another exemplary embodiment, the one or more grafts are comprised of a homogenous synthetic material, a homogenous natural material, or a combination thereof. In another exemplary embodiment, the one or more grafts are comprised of any one or more of collagen, animal xenograft, human allograft, human autograft, silicone, bioglass, peek, polyethylene, titanium, and cobalt chrome.

In another exemplary embodiment, any one of the one or more grafts includes a tapered sidewall portion disposed between a top portion and a bottom portion. In another exemplary embodiment, the tapered sidewall portion includes a diameter that decreases from a first diameter of the top portion to a second diameter of the bottom portion. In another exemplary embodiment, the tapered sidewall portion comprises a degree of tapering that is configured to prevent the graft from subsiding into a hole drilled in bone.

In another exemplary embodiment, the one or more grafts and the multiplicity of instruments are packaged together in an exterior container suitable for delivery to a practitioner. In another exemplary embodiment, the one or more grafts are stored in a first sterile container. In another exemplary embodiment, any one or more of the punch, the obturator, the guidewire, the cannulated reamer, and the insertion tamp are stored in a second sterile container. In another exemplary embodiment, the size gauge is stored in a third sterile container.

In another exemplary embodiment, wherein the size gauge is configured to indicate a suitably sized graft for treating the osteochondral/subchondral defect; and wherein the size gauge is configured to indicate a depth of an osteochondral bore drilled during treating the osteochondral/subchondral defect.

In an exemplary embodiment, a method for an osteochondral/subchondral treatment system comprises: configuring one or more grafts to treat an osteochondral/subchondral defect; configuring a size gauge to correspond to sizes of the one or more grafts; and assembling a sterile instrument kit comprising a multiplicity of instruments including any one or more of the size gauge, a punch, an obturator, a guidewire, a reamer, a cannulated reamer, a graft inserter, and an insertion tamp, the multiplicity of instruments being configured for implanting the one or more grafts into a patient's body such that the graft is flush, subflush, or slightly proud of a surrounding native cartilage surface.

In another exemplary embodiment, assembling further comprises: storing the one or more grafts in a first sterile container; storing any one or more of the multiplicity of instruments in a second sterile container; and storing the size gauge in a third sterile container. In another exemplary embodiment, configuring the one more grafts comprises forming the one or more grafts of a homogenous synthetic material, a homogenous natural material, or a combination thereof. In another exemplary embodiment, configuring the one or more grafts includes forming diameters and lengths of the one or more grafts that depend upon the particular bone joints into which the one or more grafts are to be implanted.

In an exemplary embodiment, an osteochondral implant for treating osteochondral/subchondral defects comprises: a lower portion including a bottom surface for being pressed into an osteochondral hole drilled at a defect area; and an upper portion including a top surface for replacing an osteochondral surface.

In another exemplary embodiment, at least one of the lower portion and the upper portion comprises any synthetic or natural homogenous material suitable for implantation into bone, including any one or more of collagen, animal xenograft, human allograft, human autograft, silicone, bioglass, collagen, peek, polyethylene, titanium, or cobalt chrome. In another exemplary embodiment, at least one of the lower portion and the upper portion comprises a material exhibiting a hardness of at least 30 durometer.

In another exemplary embodiment, the upper portion includes a cylindrical sidewall that extends from a periphery of the top surface to a flat undersurface. In another exemplary embodiment, the lower portion includes a cylindrical sidewall having a diameter that is substantially uniform from the undersurface to the bottom surface. In another exemplary embodiment, the lower portion includes a cylindrical sidewall having a diameter that decreases from an initial diameter at the undersurface to a bottom diameter of the bottom surface. In another exemplary embodiment, the decreasing diameter of the cylindrical sidewall is configured to prevent the implant from subsiding into the osteochondral hole.

In another exemplary embodiment, the top surface includes a positive curvature height that imparts a convex curvature to the upper portion. In another exemplary embodiment, the positive curvature height is configured to dispose the top surface slightly above cartilage tissue surrounding the defect area to be treated. In another exemplary embodiment, the top surface includes a shape configured to approximate the osteochondral or subchondral surface to be replaced.

In another exemplary embodiment, the top surface includes a positive curvature that extends to a periphery that joins an undersurface of the upper portion. In another exemplary embodiment, the undersurface extends inward from the periphery to a cylindrical sidewall comprising the lower portion. In another exemplary embodiment, the undersurface is configured to contact an exterior surface of the cartilage tissue surrounding the defect area to be treated.

In another exemplary embodiment, the lower portion is configured to be pressed into a subchondral hole such that the bottom surface contacts a bottom of the subchondral hole. In another exemplary embodiment, the upper portion includes a cylindrical sidewall configured to contact surrounding bone within the subchondral hole.

In another exemplary embodiment, the lower portion comprises a first implant material including any of a homogenous synthetic material, a homogenous natural material, or a combination thereof. In another exemplary embodiment, the first implant material comprises any one or more of collagen, animal xenograft, human allograft, human autograft, silicone, bioglass, peek, polyethylene, titanium, or cobalt chrome. In another exemplary embodiment, the upper portion comprises a second implant material configured in the form of a membrane to be placed on top of the first implant material to form a two-piece construct of the implant. In another exemplary embodiment, the second implant material comprises any one or more of collagen, human allograft membrane, animal xenograft membrane, human autograft membrane, bioglass, PGA, PLLA, Calcium phosphate, silicone, peek, polyethylene, titanium, or cobalt chrome.

In an exemplary embodiment, a method for treating an osteochondral/subchondral defect comprises: drilling a subchondral hole at a defect area of a joint; pressing a lower portion comprising a two-piece implant into the subchondral hole; and placing an upper portion comprising the two-piece implant on top of the lower portion.

In another exemplary embodiment, pressing includes using a first implant material comprising the lower portion that includes any of a homogenous synthetic material, a homogenous natural material, or a combination thereof. In another exemplary embodiment, the first implant material comprises any one or more of collagen, animal xenograft, human allograft, human autograft, silicone, bioglass, peek, polyethylene, titanium, or cobalt chrome. In another exemplary embodiment, placing includes selecting a second implant material comprising the upper portion that includes any one or more of collagen, human allograft membrane, human allograft membrane, animal xenograft membrane, bioglass, PGA, PLLA, Calcium phosphate, silicone, peek, polyethylene, titanium, or cobalt chrome.

### BRIEF DESCRIPTION OF THE DRAWINGS

The drawings refer to embodiments of the present disclosure in which:

FIG. 1 illustrates an isometric view of an exemplary embodiment of a tapered implant for treating osteochondral/subchondral defects, in accordance with the present disclosure;

FIG. 2 illustrates a side view of an exemplary embodiment of a tapered implant having a relatively wide diameter;

FIG. 3 illustrates a side view of an exemplary embodiment of a tapered implant having a relatively narrow diameter;

FIG. 4 illustrates a side plan view of the tapered implant of FIG. 2;

FIG. 5 illustrates an exemplary use environment comprising an exemplary embodiment of a tapered implant that is press-fit into an osteochondral/subchondral hole in a 1^{st} metatarsal bone;

FIG. 6 illustrates an exemplary embodiment of a sterile implant system for treating damaged cartilage joints according to the present disclosure;

FIG. 7A illustrates an exemplary embodiment of a punch that may be included in the sterile implant system of FIG. 6;

FIG. 7B illustrates the punch of FIG. 7A mounted onto a guidewire for the purpose of directing a distal blade of the punch to a damaged location within a bone joint;

FIG. 8 illustrates an isometric view of an exemplary embodiment of a tapered implant for treating osteochondral/subchondral defects, in accordance with the present disclosure;

FIG. 9 illustrates a side plan view of the tapered implant of FIG. 8, according to the present disclosure;

FIG. 10 illustrates an exemplary embodiment of a size gauge that may be incorporated into a sterile implant system for treating osteochondral/subchondral defects in damaged bone joints;

FIG. 11 illustrates an exemplary embodiment of a size gauge comprising a transparent material and configured to be incorporated into a sterile implant system for treating osteochondral/subchondral defects in damaged bone joints;

FIG. 12 illustrates an exemplary embodiment of a size gauge that may be incorporated into a sterile implant system for treating osteochondral/subchondral defects in damaged bone joints;

FIG. 13 illustrates an exemplary embodiment of a guidewire configured to indicate a depth of an instrument riding thereon;

FIG. 14 illustrates an exemplary embodiment of a cartilage punch that may be incorporated into a sterile implant system for treating osteochondral/subchondral defects in damaged bone joints;

FIG. 15 illustrates an exemplary embodiment of a cannulated obturator that is configured to cooperate with the cartilage punch of FIG. 14;

FIG. 16 illustrates an exemplary use environment wherein the cartilage punch and the cannulated obturator are being used to remove damaged articular cartilage from a bone joint being treated;

FIG. 17 illustrates a cross-sectional view of the cartilage punch and the cannulated obturator of FIG. 16 after the cartilage punch has stamped a shaped cut into the articular cartilage;

FIG. 18 illustrates an exemplary use environment wherein the cartilage punch and the cannulated obturator of FIG. 16 are directed by a guidewire and the cartilage punch has stamped a shaped cut into the articular cartilage;

FIG. 19 illustrates a cross-sectional view of the cartilage punch and the cannulated obturator directed by the guidewire of FIG. 18 after the cartilage punch has stamped a shaped cut into the articular cartilage;

FIG. 20 illustrates an exemplary embodiment of a cannulated reamer that is configured to cooperate with the cartilage punch of FIG. 14 and may be incorporated into a sterile implant system for treating osteochondral/subchondral defects in damaged bone joints;

FIG. 21 illustrates a cross-sectional view of the cannulated reamer of FIG. 20 sheathed within the cartilage punch of FIG. 14 during drilling a tapered osteochondral/subchondral bore;

FIG. 22 illustrates an exemplary embodiment of an insertion tamp that is configured to cooperate with the cartilage punch of FIG. 14 for the purpose of delivering and tamping a tapered implant into a bore drilled in a damaged bone joint;

FIG. 23 illustrates a ghost-view of the insertion tamp of FIG. 22 and a tapered implant disposed within the cartilage punch of FIG. 14 prior to tamping the implant into a bore drilled in a damaged bone joint;

FIG. 24 illustrates a ghost-view of the insertion tamp and the tapered implant disposed within the cartilage punch of FIG. 23 after the implant has been tamped to an optimal depth within the bore drilled in the damaged bone joint;

FIG. 25 illustrates an exemplary use environment wherein ring markings disposed on the insertion tamp of FIG. 22 indicate that a tapered implant has been tamped to an optimal depth within a bore drilled in a damaged bone joint;

FIG. 26 illustrates a lower perspective view of an exemplary embodiment of a graft plug kit, according the present disclosure;

FIG. 27 illustrates an upper perspective view of an exemplary embodiment of a graft plug kit in accordance with the present disclosure;

FIG. 28 illustrates a perspective view of an exemplary embodiment of a sterile instrument kit for implanting graft plugs into bone joints of a patient in accordance with the present disclosure;

FIG. 29 illustrates an isometric view of exemplary embodiment of a tapered osteochondral implant for treating osteochondral/subchondral defects in accordance with the present disclosure;

FIG. 30 illustrates a side plan view of the tapered osteochondral implant of FIG. 29;

FIG. 31 illustrates a side plan view of an exemplary embodiment of a tapered osteochondral implant having an untapered lower portion;

FIG. 32 illustrates an isometric view of exemplary embodiment of a tapered osteochondral implant for treating osteochondral/subchondral defects in accordance with the present disclosure;

FIG. 33 illustrates a side plan view of the tapered osteochondral implant of FIG. 32;

FIG. 34 illustrates a side plan view of an exemplary embodiment of a tapered osteochondral implant having an untapered lower portion;

FIG. 35 illustrates an exemplary-use environment wherein the tapered osteochondral implant of FIG. 29 is implanted into an osteochondral/subchondral hole in a 1^{st} metatarsal bone in accordance with the present disclosure; and

FIG. 36 illustrates an exemplary-use environment wherein the tapered osteochondral implant of FIG. 32 is implanted into an osteochondral/subchondral hole in a 1^{st} metatarsal bone according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first implant,” may be made. However, the specific numeric reference should not be interpreted as a literal sequential order but rather interpreted that the “first implant” is different than a “second implant.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Cartilage that facilitates articulation of the joints and protects and cushions bones can become damaged due to disease, abrupt trauma or prolonged wear. Subchondral bone that supports the cartilage can also be damaged due to disease or trauma. A number of surgical techniques have been developed to treat damaged cartilage and subchondral bone, thereby relieving pain and facilitating better joint function. One surgical technique includes transplantation of a healthy osteochondral graft to replace damaged cartilage and encourage new cartilage growth. Many grafting procedures utilize a variety of natural and synthetic tissue scaffolds, with or instead of bone, such as collagen, silicone, acrylics, hydroxyapatite, calcium sulfate, ceramics, and the like, which may be implanted into an osteochondral hole bored at a patient's defect area. As such, there is an ongoing need for the development of osteochondral/subchondral grafting capabilities for treating damage to subchondral bone and articular cartilage in joints. Provided herein are embodiments and methods for a tapered homogenous implant for treating osteochondral/subchondral defects.

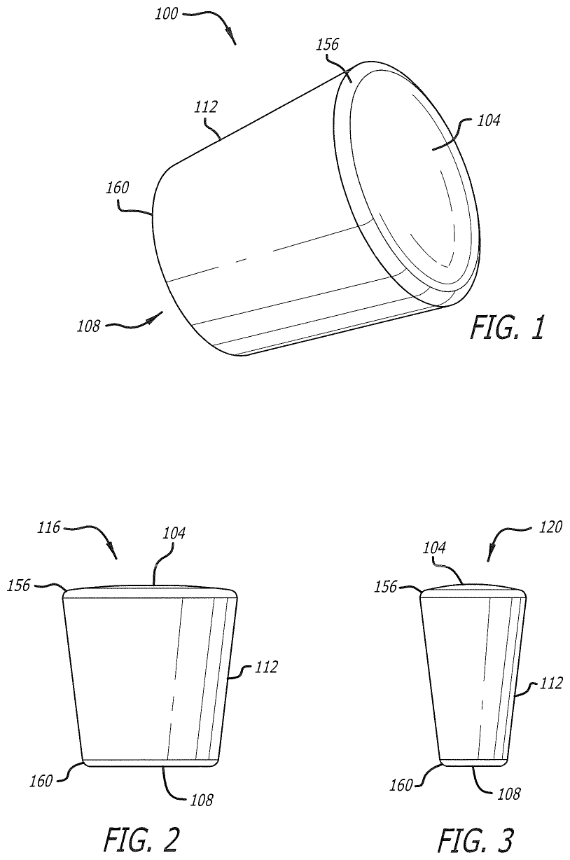

FIG. 1 illustrates an exemplary embodiment of a tapered monophasic implant 100 for treating osteochondral defects in accordance with the present disclosure. In general, the implant 100 includes a top portion 104 and a bottom portion 108 that share a cylindrical sidewall 112 extending therebetween. The implant 100 is configured to be press-fit into an osteochondral hole bored at a patient's defect area. The top portion 104 includes a shape that approximates an osteochondral surface to be replaced. The bottom portion 108 is configured to be implanted into the osteochondral hole drilled into the patient's bone. The implant 100 may comprise any synthetic or natural homogenous material suitable for implantation into bone, including any one or more of collagen, human allograft or autograft, animal xenograft, silicone, bioglass, collagen, peek, polyethylene, titanium, cobalt chrome, and the like. In some embodiments, the implant 100 is comprised of a material exhibiting a hardness of at least 30 durometer.

As shown in FIGS. 2-3, the implant 100 may be implemented with a range of diameters that facilitate using the implant 100 to treat osteochondral or subchondral defects in various bone joint locations in the human body, such as by way of non-limiting example, a femoral condyle, a humeral head, a talus, the trapezium of the hand, the capitellum of the elbow, as well as any of the metatarsal and phalangeal joints of the hand or foot. As such, FIG. 2 illustrates a side view of an exemplary embodiment of a tapered monophasic implant 116 having a relatively wide diameter, and FIG. 3 shows a side view of an exemplary tapered monophasic implant 120 having a relatively narrow diameter. It is contemplated, however, that the overall size of the implant 100 is to be selected according to the particular bone joint to be treated.

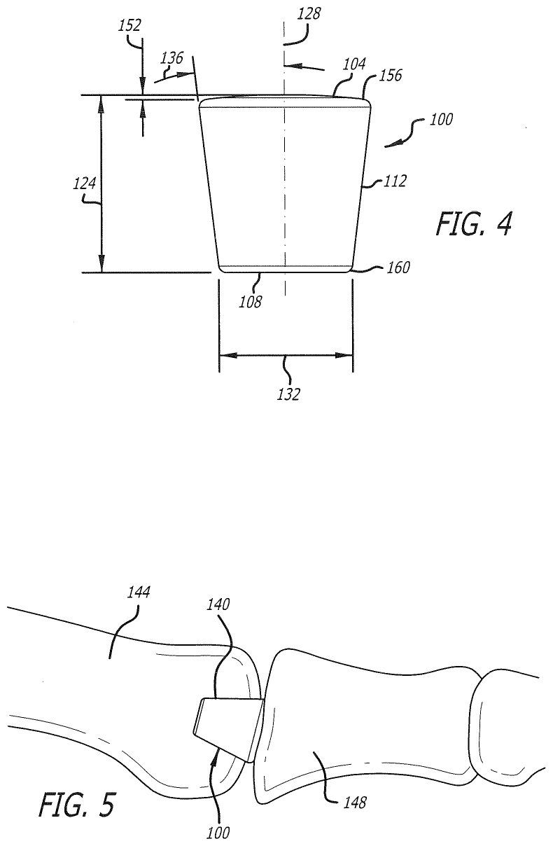

As best shown in FIG. 4, the implant 100 possesses a height 124 along a longitudinal axis 128 of the implant and a bottom diameter 132 centered on the longitudinal axis 128. The height 124 extends from the bottom portion 108 to the highest region of the top portion 104, such as the region of the top portion 104 around the longitudinal axis 128. In one embodiment, the height 124 is substantially 10 millimeters (mm). It is contemplated, however, that the height 124 may be varied according to the bone joint to be treated, and thus the implant 100 may be implemented with a wide variety of heights 124, without limitation.

The cylindrical sidewall 112 of the implant 100 includes a taper that causes a diameter of the sidewall 112 to decrease from a diameter of the top portion 104 to the bottom diameter 132 of the bottom portion 108. As shown in FIG. 4, the taper of the sidewall 112 may be expressed in terms of a taper half-angle 136 taken with respect to the longitudinal axis 128. The taper of the sidewall 112 is configured to prevent the implant 100 from subsiding into the osteochondral hole drilled in bone. As will be appreciated, therefore, the taper half-angle 136 may be any angle that is found to prevent subsidence of the implant 100, without limitation. Accordingly, it is contemplated that in some embodiments, the taper half-angle 136 is substantially zero degrees. In such embodiments, the diameter of the top portion 104 is substantially the same as the bottom diameter 132 of the bottom portion 108, and thus the cylindrical sidewall 112 comprises a straight cylindrical shape, without limitation.

In some embodiments, the overall size of the implant 100 is identified based on the bottom diameter 132 without a specific reference to the included taper half-angle 136 of the implant 100. In such embodiments, a practitioner may select the implant 100 based on a size of the osteochondral hole to be drilled into the patient's bone. As with other dimensions of the implant 100 discussed hereinabove, however, the bottom diameter 132 may be varied according to the bone joint to be treated. In one embodiment, the bottom diameter 132 ranges between substantially 5 mm and substantially 10 mm. As will be appreciated, therefore, the implant 100 may be implemented with a wide variety of bottom diameters 132, without limitation.

In some embodiments, the overall size of the implant 100 may be identified based on the diameter of the top portion 104, and thus the size of the implant 100 may be selected based on the area of the joint defect to be treated. It is contemplated that in such embodiments, the specific sizes of the bottom diameter 132 and the taper half-angle 136 may be incorporated into the implant 100 in accordance with the diameter of the top portion 104, and thus the sizes of the bottom diameter 132 and the taper half-angle 136 need not be specifically called out. For example, in some embodiments, any one or more of the height 124, the taper half-angle 136, and the bottom diameter 132 of the implant 100 may be configured to correlate with the diameter of the top portion 104, without limitation.

FIG. 5 illustrates an exemplary use environment wherein the tapered monophasic implant 100 is implanted into an osteochondral hole 140 drilled in a 1^{st} metatarsal bone 144. As will be recognized, the top portion 104 of the implant 100 is disposed slightly above the surrounding cartilage tissue of the 1^{st} metatarsal bone 144 and in contact with an adjacent 1^{st} proximal phalangeal bone 148. In general, the top portion 104 includes a shape configured to approximate the osteochondral surface to be replaced. In some embodiments, such as the illustrated embodiment of FIG. 5, the shape of the top portion 104 (see FIG. 4) includes a convex curvature that approximates the curvature of the osteochondral surface to be replaced. In embodiments of the top portion 104 including a convex curvature, the implant 100 includes a positive curvature height 152 as shown in FIG. 4. In some embodiments, the top portion 104 includes a concave curvature that corresponds to a negative curvature height 152 of the implant 100. It is contemplated that an embodiment of the implant 100 including a negative curvature height 152 is advantageously configured for treating cartilage defects in the 1^{st} proximal phalangeal bone 148.

As further shown in FIG. 5, the implant 100 includes a height 124 (see FIG. 4) that places the bottom portion 108 in contact with a bottom of the osteochondral hole 140 and elevates the top portion 104 slightly above the surrounding cartilage tissue of the 1^{st} metatarsal bone 144. The taper half-angle 136 advantageously prevents subsidence of the implant 100 into the osteochondral hole 140, even in the event that the bone below the bottom portion 108 subsides. As best illustrated in FIG. 4, the implant 100 includes a rounded periphery 156 that joins the top portion 104 and the cylindrical sidewall 112. The rounded periphery 156 comprises a transition surface between the top portion 104 and the sidewall 112 that provides a smooth contact surface to surrounding tissues. Further, the implant 100 includes a rounded periphery 160 that joins the cylindrical sidewall 112 and the bottom portion 108. As will be appreciated, the rounded periphery 160 provides a smooth transition surface between the sidewall 112 and the bottom portion 108 that prevents damage to the interior sidewalls of the osteochondral hole 140 during insertion of the implant 100 therein.

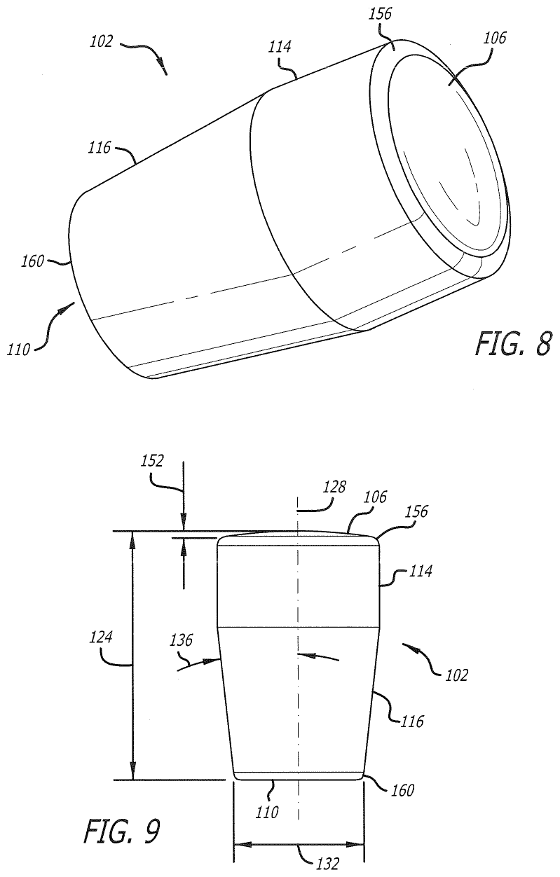

FIG. 8 illustrates an exemplary embodiment of a tapered monophasic implant 102 for treating osteochondral/subchondral defects in accordance with the present disclosure. The implant 102 is similar to the implant 100, shown in FIG. 1, with the exception that the implant 102 includes an untapered cylindrical sidewall 114 adjacent to a top portion 106. A tapered cylindrical sidewall 116 extends from the untapered cylindrical sidewall 114 to a bottom portion 110, as shown in FIG. 8. Like the implant 100, the implant 102 is configured to be press-fit into an osteochondral hole bored at a patient's defect area. The top portion 106 includes a shape that approximates an osteochondral surface to be replaced while the bottom portion 110 is configured to be implanted into the osteochondral hole drilled into the patient's bone. The implant 102 may comprise any synthetic or natural homogenous material suitable for implantation into bone, including any one or more of collagen, human allograft or autograft, animal xenograft, silicone, bioglass, collagen, peek, polyethylene, titanium, cobalt chrome, and the like. It is contemplated that, in some embodiments, the implant 102 is comprised of a material exhibiting a hardness of at least 30 durometer.

In general, the implant 102 may be implemented with a range of diameters, heights, and tapers that facilitate using the implant 102 to treat osteochondral or subchondral defects in various bone joint locations in the human body, such as by way of non-limiting example, a femoral condyle, a humeral head, a talus, the trapezium of the hand, the capitellum of the elbow, as well as any of the metatarsal and phalangeal joints of the hand or foot. As such, the implant 102 may include any of various overall diameters ranging from a relatively wide diameter to a very narrow diameter, according to the particular bone joint to be treated.

As shown in FIG. 9, the height 124 of the implant 102 generally extends from the bottom portion 110, along the longitudinal axis 128 of the implant 102 to the highest region of the top portion 106. The height 124 is configured to place the bottom portion 110 in contact with a bottom of a hole drilled in a bone, such as the osteochondral hole 140, and elevate the top portion 106 slightly above the surrounding cartilage tissue of the bone, such as the 1^{st} metatarsal bone 144. It is contemplated, however, that the height 124 may be varied according to the bone joint to be treated, and thus the implant 102 may be implemented with a wide variety of heights 124, without limitation. In one embodiment, for example, the height 124 is substantially 10 mm.

With continuing reference to FIG. 9, the tapered cylindrical sidewall 116 includes a diameter that decreases from a diameter of the cylindrical sidewall 114 to a bottom diameter 132 of the bottom portion 110. As described hereinabove with respect to FIG. 4, the decreasing diameter of the tapered cylindrical sidewall 116 may be expressed in terms of a taper half-angle 136 taken with respect to the longitudinal axis 128. The taper of the cylindrical sidewall 116 generally is configured to prevent the implant 102 from subsiding into an osteochondral hole drilled in bone, such as the osteochondral hole 140, even in the event that the bone below the bottom portion 110 subsides. As such, the taper half-angle 136 may be any angle, including an angle of zero degrees, that is found to prevent subsidence of the implant 102, without limitation.

Moreover, the cylindrical sidewall 114 generally comprises a straight cylindrical shape that includes a taper half-angle 136 of substantially zero degrees, without limitation. Thus, the cylindrical sidewall 114 shares the same diameter as the diameter of the top portion 106. In some embodiments, however, the cylindrical sidewall 114 may include a non-zero taper half-angle that differs from the taper half-angle 136 of the sidewall portion 116. In such embodiments, the diameter of the cylindrical sidewall 114 decreases from the diameter of the top portion 106 to the diameter of a top of the cylindrical sidewall 116, and the diameter of the cylindrical sidewall 116 decreases from the diameter of the top of the cylindrical sidewall 116 to the bottom diameter 132 of the bottom portion 110. Expressed equivalently, the sidewall 114 may include a first taper half-angle and the sidewall 116 may include a second taper half-angle 136, wherein the second taper half-angle 136 is greater than the first taper half-angle.

In some embodiments, the overall size of the implant 100 is identified based on the bottom diameter 132 without a specific reference to the included taper half-angle 136 of the implant 102. In such embodiments, a practitioner may select the implant 102 based on a size of the osteochondral hole to be drilled into the patient's bone. As with other dimensions of the implant 102 discussed hereinabove, however, the bottom diameter 132 may be varied according to the bone joint to be treated. In one embodiment, the bottom diameter 132 ranges between substantially 5 mm and substantially 10 mm. As will be appreciated, therefore, the implant 102 may be implemented with a wide variety of bottom diameters 132, without limitation.

Similar to the implant 100, described above, the overall size of the implant 102 may be identified based on the diameter of the top portion 106 so as to enable selecting the implant 102 based on the area of the joint defect to be treated. In such embodiments, the specific sizes of the bottom diameter 132 and the taper half-angle 136 may be incorporated into the implant 102 in accordance with the diameter of the top portion 106, and thus the sizes of the bottom diameter 132 and the taper half-angle 136 need not be specifically called out. For example, in some embodiments, any one or more of the height 124, the taper half-angle 136, a height of the sidewall 114, a height of the sidewall 116, a non-zero taper half-angle of the sidewall 114 (where applicable), and the bottom diameter 132 of the implant 102 may be configured to correlate with the diameter of the top portion 106, without limitation.

As further shown in FIG. 9, the top portion 106 includes a positive curvature height 152 that imparts a convex curvature to the implant 102. As will be recognized, the positive curvature height 152 may be used to dispose the top portion 106 of the implant 102 slightly above surrounding cartilage tissue of the bone to be treated. In general, however, the top portion 106 includes a shape configured to approximate the osteochondral or subchondral surface to be replaced. For example, in some embodiments, the shape of the top portion 106 includes a curvature that approximates the curvature of the osteochondral surface to be replaced. As such, in some embodiments, the top portion 106 includes a concave curvature that corresponds to a negative curvature height 152 of the implant 102. It is contemplated that an embodiment of the implant 102 that includes a negative curvature height 152 may be advantageously configured for treating cartilage defects in the 1^{st} proximal phalangeal bone, while an embodiment of the implant 102 that includes a positive curvature height 152 may be configured for treating cartilage defects in the 1^{st} metatarsal bone. For subchondral implants, the top surface may have a flat curvature as the implant generally is disposed below the surrounding articular surface and thus does not need to approximate the shape of articular surface.

As further shown in FIG. 9, a rounded periphery 156 joins the top portion 106 with the cylindrical sidewall 114. The rounded periphery 156 comprises a transition surface between the top portion 106 and the cylindrical sidewall 114 that provides a smooth contact surface to surrounding tissues. Similarly, a rounded periphery 160 joins the cylindrical sidewall 116 and the bottom portion 110 of the implant 102. As will be appreciated, the rounded periphery 160 provides a smooth transition surface between the cylindrical sidewall 116 and the bottom portion 110 that prevents damage to the interior sidewalls of a hole drilled in bone, such as the osteochondral hole 140, during insertion of the implant 102 therein.

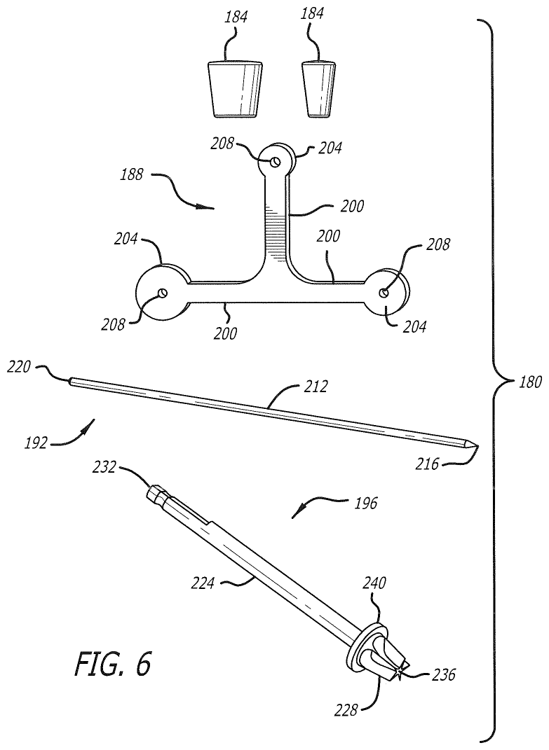

Turning, now, to FIG. 6, an exemplary embodiment of a sterile implant system 180 is shown for treating osteochondral/subchondral defects according to the present disclosure. In the embodiment illustrated in FIG. 6, the sterile implant system 180 comprises one or more osteochondral/subchondral implants 184, a size gauge 188, a guidewire 192, and a cannulated reamer 196. In some embodiments, the sterile implant system 180 may further comprise a graft inserter and/or a tamp, as described herein. As will be appreciated, the sterile implant system 180 comprises instruments necessary for treating osteochondral/subchondral defects by way of surgery. The sizes of the instruments comprising the implant system 180 will depend upon the size of the particular implant 184 to be implanted into the patient. It is envisioned, therefore, that a surgeon may select the implant 184 and a correspondingly sized embodiment of the implant system 180 based on the location and size of the bone joint to be treated.

With continuing reference to FIG. 6, the size gauge 188 comprises multiple tabs 200, each of which representing a particular size of the implant 184. Each of the multiple tabs 200 includes a circular portion 204 having a central hole 208. The circular portions 204 approximate the areas of different implants 184, and the central hole 208 has a diameter suitable to receive the guidewire 192. As will be appreciated by those skilled in the art, the circular portions 204 facilitate identifying an advantageously sized implant 184 for treating the damaged bone joint. The central hole 208 facilitates inserting the guidewire 192 through the size gauge 188.

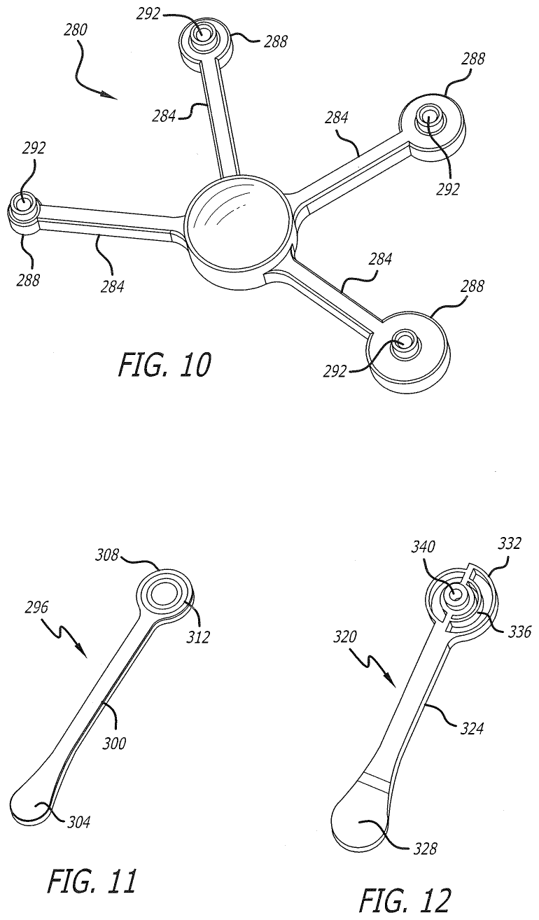

The instruments comprising the sterile implant system 180 are not to be limited to the specific instruments, or the sizes and shapes of the instruments shown in FIG. 6. For example, FIG. 10 illustrates an exemplary embodiment of a size gauge 280 that may be incorporated into the sterile implant system 180, without limitation. Similar to the size gauge 188, the size gauge 280 includes multiple arms 284 that each extends to a circular portion 288 having a central hole 292. The circular portions 288 approximate the areas of different implants 184, and the central holes 292 have a diameter suitable to receive the guidewire 192. The circular portions 288 enable the surgeon to identify a suitably sized implant 184 for treating the damaged bone joint. The central hole 292 facilitate inserting the guidewire 192 through the size gauge 280 to identify the center of the damaged area of the bone joint to be treated. As will be appreciated, the size gauge 280 provides four circular portions 288 corresponding to different sizes of implants 184 that may be used to treat osteochondral/subchondral defects.

FIG. 11 illustrates an exemplary embodiment of a size gauge 296 that may be included in the sterile implant system 180 of FIG. 6, without limitation. The size gauge 296 is a generally elongate member 300 including a proximal handle 304 and a distal circular portion 308. The size gauge 296 preferably is comprised of a transparent material, such as biocompatible plastic, that enables observation of damaged bone joint areas while the size gauge 296 is positioned over or near bone joints. Further, the size gauge 296 includes multiple delineation rings 312 concentrically disposed on the circular portion 308. The circular portion 308 and the delineation rings 312 enable the surgeon to identify an advantageously sized implant 184 to treat the damaged area being viewed through the circular portion 308 of the size gauge 296. In the illustrated embodiment, three delineation rings 312 and the area of the circular portion 308 are configured to correspond to four different sizes of implants 184. In some embodiments, more than or less than three delineation rings 312 may be incorporated into the size gauge 296, without limitation. Further, in some embodiments, a central hole may be concentrically disposed in the circular portion 308 so as to facilitate inserting the guidewire 192 through the size gauge 296 to identify the center of the damaged area of the bone joint to be treated.

FIG. 12 illustrates an exemplary embodiment of a size gauge 320 that may be included in the sterile implant system 180 of FIG. 6, without limitation. Similar to the size gauge 296 of FIG. 11, the size gauge 320 is a generally elongate member 324 that includes a proximal handle 328 and a distal circular portion 332. The circular portion 332 comprises multiple circular area delineators 336 and a central hole 340 that are concentrically disposed within the circular portion 332. In the illustrated embodiment, four circular area delineators 336 are configured to correspond to different sizes of implants 184. In some embodiments, more than or less than four circular area delineators 336 may be incorporated into the size gauge 320, without limitation. Open space between adjacent circular area delineators 336 enables observation of damaged bone joint areas while the size gauge 320 is positioned over or near bone joints. The circular area delineators 336 enable the surgeon to identify an advantageously sized implant 184 to treat the damaged area being viewed through the open spaces between circular area delineators 336 of the size gauge 320. The central hole 340 facilitates inserting the guidewire 192 through the size gauge 320 to identify the center of the damaged area of the bone joint to be treated.

With reference, again, to FIG. 6, the guidewire 192 comprises an elongate shaft 212 having a distal pointed tip 216 and a proximal blunt end 220. The guidewire 192 is configured to be inserted into confined spaces within bone joints and serves to direct a subsequent insertion of the cannulated reamer 196 to the implant location within the bone joint. In some embodiments, the guidewire 192 is comprised of a surgical stainless steel, such as austenitic 316 stainless steel, martensitic 440 stainless steel, martensitic 420 stainless steel, and the like. It will be appreciated that the distal pointed tip 216 facilitates advancing the guidewire 192 through obstructive tissues and structures, and the proximal blunt end 220 facilitates manipulating the guidewire 192 by hand, or by way of an appropriate tool.

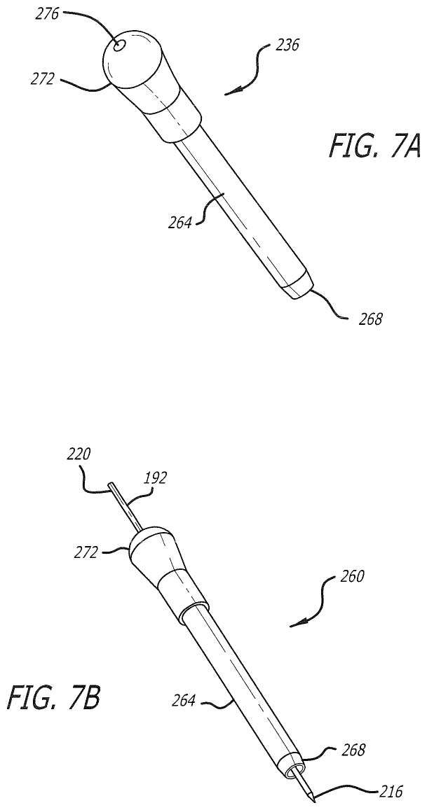

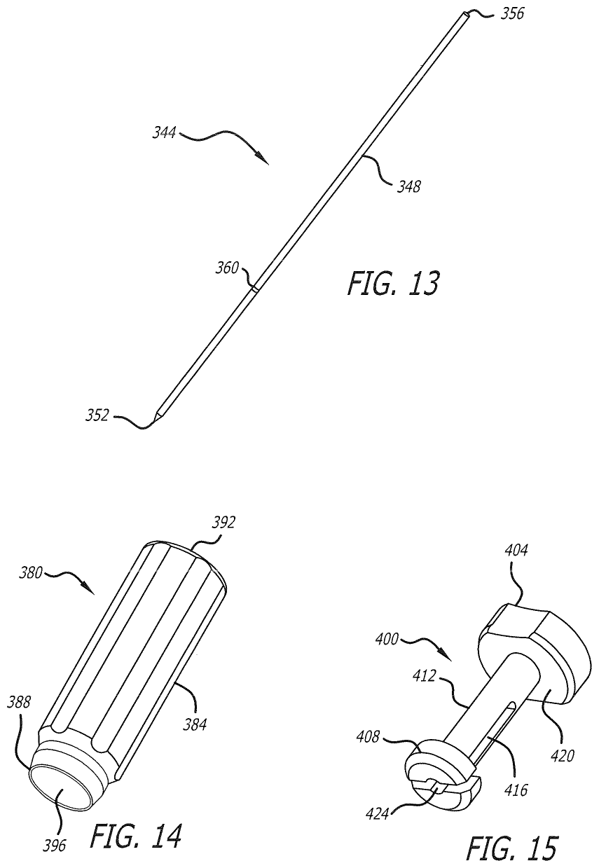

As mentioned hereinabove, the instruments comprising the sterile implant system 180 are not to be limited to the specific instruments shown in FIG. 6. For example, FIG. 13 illustrates an exemplary embodiment of a guidewire 344 that may be included in the sterile implant system 180 of FIG. 6, without limitation. The guidewire 344 comprises an elongate shaft 348 having a trocar tip 352 and a proximal blunt end 356. Similar to the guidewire 192 of FIG. 6, the guidewire 344 of FIG. 13 is configured to enable the surgeon to identity the center of a damaged area of a bone joint to be treated and serves to direct subsequent insertion of instruments to the implant location within the bone joint. A depth indicator 360 is disposed along the elongate shaft 348 and configured to indicate the depth to which the guidewire 344 is inserted into the bone joint to be treated. In some embodiments, the depth indicator 360 may be configured to indicate a depth of an instrument riding on the guidewire 344. For example, a punch 260 (see FIGS. 7A-7B) may be directed along the guidewire 344 to the damaged area to be treated, and the depth indicator 356 may be used to identify the depth to which the punch 260 is to be pushed into the joint to perform a suitable cut into the cartilage of the joint. As such, it is contemplated that any number of indicators 360 may be disposed along the length of the guidewire 344 in any of various desired locations corresponding to any of various instruments that may be directed by way of the guidewire 344 into bone joints to be treated, without limitation.

With reference, again, to FIG. 6, the cannulated reamer 196 comprises a rigid elongate shaft 224 having a distal cutting end 228 and a proximal shank 232. The distal cutting end 228 comprises a cutting edge suitable for rotatably clearing a tapered osteochondral bore, thereby removing damaged articular cartilage and an underlying bone portion from the bone joint being treated. In some embodiments, the distal cutting edge 228 comprises a spiral cutting edge, although other suitable cutting edge configurations will be apparent. The proximal shank 232 is configured to be grasped by a chuck of a surgical drill, or other equivalent rotary tool. Further, in some embodiments the cannulated reamer 196 comprises a central, lengthwise hole 236 whereby the reamer may be mounted onto the guidewire 192 so as to direct the distal cutting end 228 to the damage location within the bone joint. A peripheral disc 240 is configured to operate as a depth gauge. As will be appreciated, the disc 240 coming into contact with tissue surround a bore being drilled operates as an indication that the bore has an optimal depth to receive the tapered implant 184.

It is contemplated that, in some embodiments, the distal cutting edge 228 includes a tapered diameter that corresponds to the tapered diameter of the implant 184, as described herein. In general, the shape and size of the distal cutting edge 228 included in the instrument kit 180 corresponds the shape and size of the particular implant 184 included in the kit, as well as being indicated by at least one of the circular portions 204 of the size gauge 188. Thus, it is contemplated that the surgeon may use the size gauge 188 to select an advantageously sized implant 184 to replace damaged cartilage in the bone joint, and then extend the guidewire 192 through the central hole 208 to locate a center of the bore to be drilled. With the size of the implant 184 known, the surgeon may remove the size gauge 188 from the guidewire 192 and then extend an appropriately sized cannulated reamer 196 along the guidewire 192 to the site of the damaged cartilage to be removed. Other surgery techniques will be apparent to those skilled in the art.

FIG. 7A illustrates an exemplary embodiment of a punch 260 that may, in some embodiments, be included in the sterile implant system 180 shown in FIG. 6. The punch 260 comprises a generally elongate member 264 having a distal punch blade 268 and a rounded proximal handle 272. The distal punch blade 268 comprises a cutting edge suitable for stamping a shaped cut into the cartilage prior to drilling with the cannulated reamer 196 as described above. The shaped cut facilitates removing damaged articular cartilage from the bone joint being treated. In some embodiments, the distal punch blade 268 is circular, and thus enables stamping a circular cut in the cartilage. Shapes other than circular are contemplated, however, such as, by way of non-limiting example, any of various generally circular, oval, round, or other closed perimeter shapes, and the like, without limitation. The rounded proximal handle 272 is configured to be grasped by hand for pushing the distal punch blade 268 into the cartilage for cutting purposes. Further, the punch 260 comprises a central, lengthwise hole 276. As best shown in FIG. 7B, the hole 276 enables the punch 260 to be mounted onto the guidewire 192 so as to direct the distal punch blade 268 to the damage location within the bone joint.

FIG. 14 illustrates an exemplary embodiment of a cartilage punch 380 that may, in some embodiments, be included in the sterile implant system 180 of FIG. 6. The punch 380 comprises a generally cylindrical member 384 having a distal punch blade 388 and a proximal blunt end 393. The distal punch blade 388 is substantially similar to the distal punch blade 268, described in connection with FIGS. 7A-7B. As such, the distal punch blade 388 comprises a cutting edge suitable for stamping a shaped cut into cartilage during treatment of a damage bone joint, as described herein. The shaped cut facilitates removing damaged articular cartilage from the bone joint being treated. The proximal bunt end 392 is configured to cooperate with various instruments that may be inserted through a central hole 396 of the cartilage punch 380 during treating the damaged bone joint, as described herein.

FIG. 15 illustrates an exemplary embodiment of a cannulated obturator 400 that is configured to cooperate with the cartilage punch 380 of FIG. 14. The cannulated obturator 400 includes a proximal handle 404 and a distal gripping portion 408 that are interconnected by way of a shaft 412. The distal gripping portion 408 comprises a disc-shaped member having a diameter suitable to slidably contact an interior surface of the central hole 396 of the cartilage punch 380. A slot 416 that bisects the distal gripping portion 408 and a portion of the shaft 412 imparts a degree of flexibility to the distal gripping portion 408, such that the distal gripping portion 408 presses against the interior of the central hole 396 with a mild contact force. It is contemplated that the mild contact force is sufficient to retain the cannulated obturator 400 within the central hole 396 and allows for removal of the cannulated obturator 400 from the central hole 396 without undue effort.

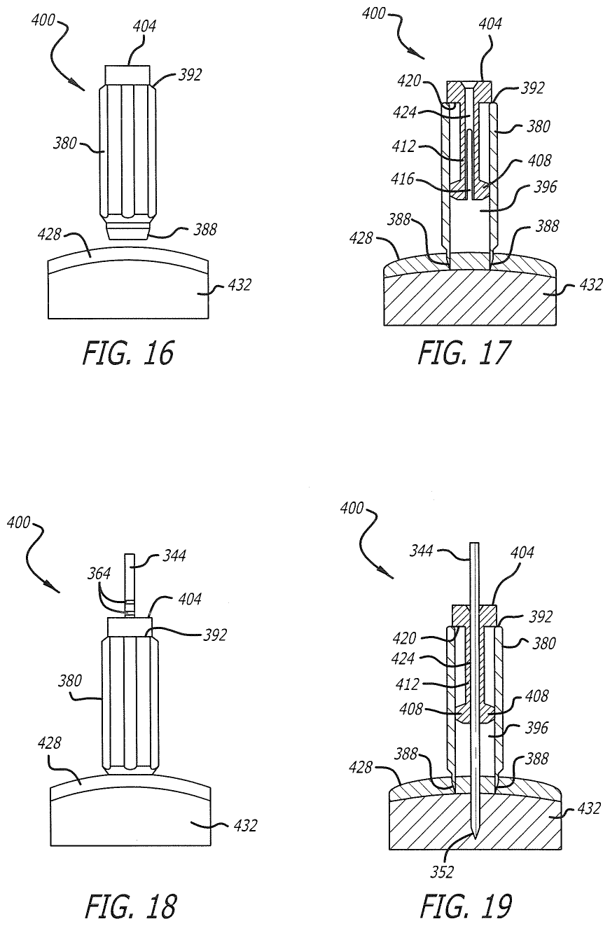

The proximal handle 404 includes an interference surface 420 that surrounds the shaft 412 and is configured to contact the proximal blunt end 392 of the cartilage punch 380, as shown in FIGS. 16-17. The interference surface 420 serves to limit the depth to which the cannulated obturator 400 may be inserted into the central hole 396 of the cartilage punch 380. The cannulated obturator 400 further includes a lengthwise hole 424 configured to receive the guidewire 344. As will be appreciated, with the cannulated obturator 400 inserted into the central hole 396, the cartilage punch 380 may be directed along the guidewire 344 to the damaged bone joint by way of the lengthwise hole 424, as shown in FIGS. 18-19.

FIGS. 16 and 17 illustrate an exemplary use environment wherein the cartilage punch 380 and the cannulated obturator 400 are being used to remove damaged articular cartilage 428 from a bone joint 432 being treated. In the exemplary use environment of FIGS. 16-17, the cannulated obturator 400 is disposed in the central hole 396 of the cartilage punch 380 such that the interference surface 404 is in contact with the distal blunt end 392. As such, the proximal handle 404 may be used to apply a cutting force to the cartilage punch 380, such that the distal cutting blade 388 stamps a shaped cut into cartilage 428, as shown in FIG. 17. The shaped cut facilitates removing the damaged articular cartilage 428 from the bone joint being treated.

FIGS. 18 and 19 illustrate an exemplary use environment wherein the cartilage punch 380 and the cannulated obturator 400 are being used in combination with the guidewire 344 to remove damaged articular cartilage 428 from the bone joint 432. The exemplary use environment shown in FIGS. 18-19 is substantially similar to the exemplary use environment of FIGS. 16-17, with the exception that in the exemplary use environment of FIGS. 18-19, the guidewire 344 is being use to guide the cartilage punch 380 and the cannulated obturator 400 by way of the lengthwise hole 424 of the obturator 400. As best shown in FIG. 18, one or more depth indicators 360 disposed along the guidewire 344 may be used to indicate the depth of the distal cutting blade 388 in the articular cartilage 428 during stamping the shaped cut. For example, in the embodiment shown in FIG. 18, the proximal handle 404 may be aligned with a first depth indicator 364 before stamping the articular cartilage 428. During stamping, however, alignment of the depth indicator 360 with the proximal handle 404 indicates that the distal cutting blade 388 has been optimally pressed into the articular cartilage 428, as shown in FIG. 19. It is contemplated, therefore, that any number of depth indicators 360 may be disposed along the length of the guidewire 344 in any of various desired locations corresponding to any of various instruments that may be directed by way of the guidewire 344 into bone joints to be treated, without limitation.

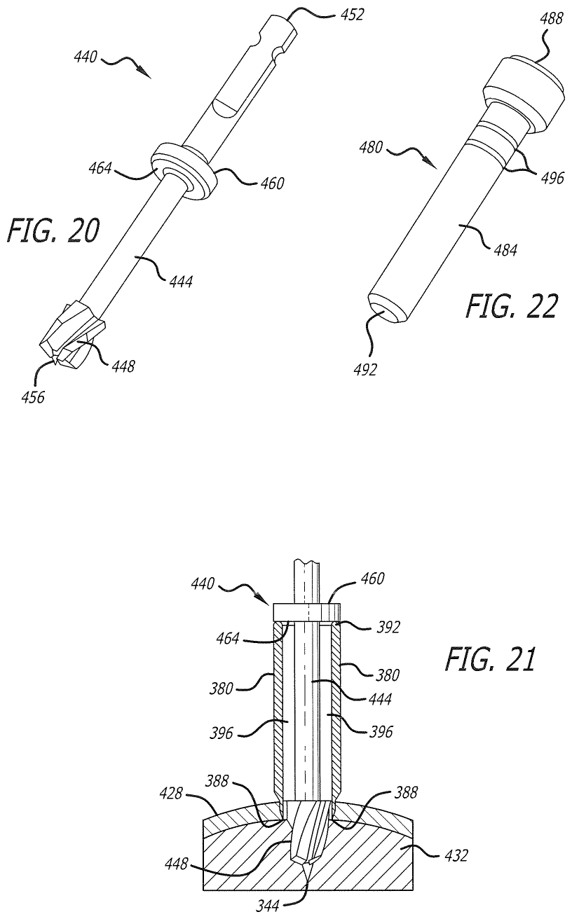

FIG. 20 illustrates an exemplary embodiment of a cannulated reamer 440 that is configured to cooperate with the cartilage punch 380 of FIG. 14. The cannulated reamer 440 comprises a rigid elongate shaft 444 having a distal cutting end 448 and a proximal shank 452. The distal cutting end 448 comprises a cutting edge suitable for rotatably clearing a tapered osteochondral/subchondral bore, thereby removing damaged articular cartilage and an underlying bone portion from the bone joint being treated. In some embodiments, the distal cutting edge 448 comprises a spiral cutting edge, although other suitable cutting-edge configurations are envisioned. The proximal shank 452 is configured to be grasped by a chuck of a surgical drill, or other equivalent rotary tool. Further, the cannulated reamer 440 comprises a central, lengthwise hole 456 whereby the reamer may be mounted onto the guidewire 344 so as to direct the distal cutting end 448 to the damaged location within the bone joint.

In the embodiment shown in FIG. 20, the cannulated reamer 440 includes a positive stop 460 comprising an interference surface 464. The interference surface 464 is a flat surface that surrounds the elongate shaft 444 and is configured to contact the proximal blunt end 392 of the cartilage punch 380 during drilling a tapered osteochondral/subchondral bore. As shown in FIG. 21, for example, the cannulated reamer 440 may be directed to the damaged bone joint by way of the guidewire 344 and sheathed within the cartilage punch 380. It is contemplated that sheathing the cannulated reamer 440 within the cartilage punch 380 serves to prevent damage to nearby tissue during navigating the distal cutting edge 448 to the damaged bone site. It is further contemplated that contact between the interference surface 464 and the proximal blunt end 392 may operate as a depth gauge during drilling the bone 432. To this end, contact between the interference surface 464 and the proximal blunt end 392 limits cutting too deeply into the bone 432 and thus serves as an indication to the surgeon that drilling may be ceased.

In some embodiments, the distal cutting edge 448 includes a tapered diameter that corresponds to the tapered diameter of the implant 184, as described herein. Further, in some embodiments, wherein the implant 184 resembles the implant 102, shown in FIGS. 8-9, the distal cutting edge 448 may include a portion having an untapered diameter that matches the untapered cylindrical sidewall 114 of the implant 102. In general, the shape and size of the distal cutting edge 448 included in the sterile implant system 180 corresponds the shape and size of the particular implant 184 included in the system 180, as well as being indicated by the accompanying size gauge included in the system 180, such as the size gauge 280 shown in FIG. 10.

FIG. 22 illustrates an exemplary embodiment of an insertion tamp 480 that is configured to cooperate with the cartilage punch 380 of FIG. 14 for the purpose of delivering and tamping the implant 184 into a bore drilled in a damaged bone joint. The insertion tamp 480 is a generally elongate member 484 including a proximal handle 488 and a distal flat surface 492. The proximal handle 488 is configured to receive a distally directed force suitable for tamping the implant 184 into the bore. The distal flat surface 492 is configured to convey the distally directed force to the implant 184 without damaging the implant 184. The elongate member 484 preferably has diameter suitable for sliding within the central hole 396 of the cartilage punch 380 without undue friction. Further, one or more ring markings 496 may be disposed on the elongate member 484 and configured to cooperate with the cartilage punch 380 to indicate the depth to which the implant 184 is tamped into the bore.

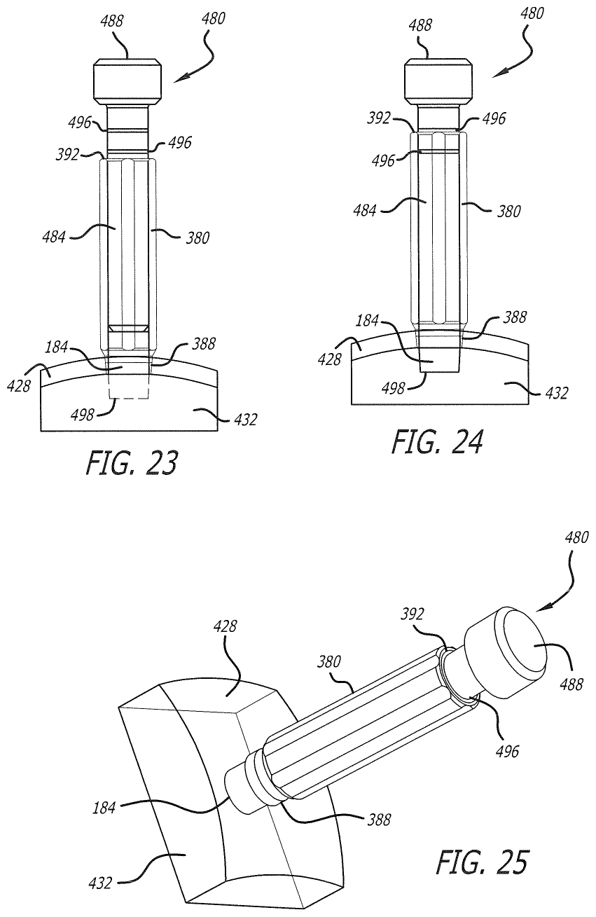

FIGS. 23-25 illustrate an exemplary use environment wherein the insertion tamp 480 is being used in combination with the cartilage punch 380 to tamp an implant 184 into a bore 498 to treat a damaged bone joint. As best shown in FIG. 23, upon inserting the implant 184 and the insertion tamp 480 into the cartilage punch 380, but before tamping the implant 184 into the bore 498, a lower ring marking 496 remains visible above the proximal blunt end 392 of the punch 380. As shown in FIGS. 24 and 25, however, upon using the proximal handle 488 of the insertion tamp 480 to optimally tamp the implant 184 into the bore 498, an upper ring marking 496 remains visible above the proximal blunt end 392. As mentioned above, the ring markings 496 may be configured to cooperate with the proximal blunt end 392 of the cartilage punch 380 to indicate an optimal depth to which the implant 184 is tamped into the bore 498. It is contemplated that configuring the ring markings 496 to indicate the optimal depth, as best shown in FIG. 25, will help the surgeon to avoid insufficiently tamping the implant 184 into the bore 498 as well as tamping the implant 184 too deeply into the bore 498.

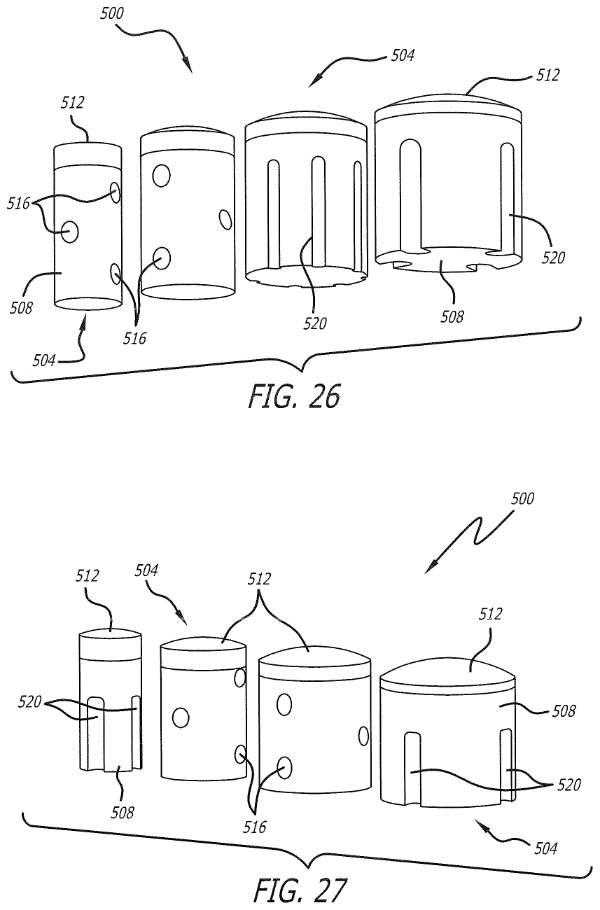

FIGS. 26 and 27 illustrate respective lower and upper perspective views of exemplary embodiments of a sterile plug system 500 advantageously configured for repairing a wide range of osteochondral defects, according the present disclosure. The sterile plug system 500 generally comprises a multiplicity of grafts 504 ranging from a relatively small diameter to a relatively large diameter. It will be appreciated that the range in diameters facilitates using the sterile plug system 500 to treat osteochondral defects in various bone joint locations in the human body, such as by way of non-limiting example, a femoral condyle (most common), a humeral head, a talus, a capitellum of the elbow, and the like. Further, the grafts 504 may be configured similarly to the implants 100, 102, respectively shown in FIGS. 1 and 8. For example, in some embodiments, the grafts 504 may include an untapered cylindrical sidewall 114 adjacent to a top portion 106, as shown in FIG. 8, and a tapered cylindrical sidewall 116 that extends from the untapered cylindrical sidewall 114 to a bottom portion 110. Like the implants 100, 102, the grafts 504 may be configured to be press-fit into an osteochondral hole bored at a patient's defect area. As such, any one or more of the grafts 504 may be incorporated into the sterile implant system 180 and comprise the implants 184, without limitation.

In the exemplary embodiments illustrated in FIGS. 26 and 27, the sterile plug system 500 comprises four grafts 504 ranging in size from substantially 5 millimeters (mm) in diameter to substantially 15 mm in diameter. In some embodiments, the sterile plug system 500 may comprise a number of grafts greater than four, and thus grafts having diameters smaller than 5 mm and/or greater than 15 mm may be included in the graft plug kit 100. Moreover, the grafts 504 in the embodiments illustrated in FIGS. 26 and 27 each comprises a length of substantially 12 mm. In some embodiments, however, the grafts 504 may comprise different lengths, depending upon the particular bone joints for which the grafts 504 are intended. In some embodiments, the lengths of the grafts 504 may range from a relatively small value to a relatively large value. In some embodiments, the length of each graft 504 may be configured to correlate with the diameter of the graft. It will be appreciated that the sterile plug system 500 advantageously provides specifically sized grafts 504 whereby a surgeon may select the grafts based on a particular bone joint to be treated. Further, it should be understood that a wide variety of dimensions and sizes of the grafts 504 may be incorporated into the sterile plug system 500 without deviating from the spirit and scope of the present disclosure.

As further illustrated in FIGS. 26 and 27, each of the grafts 504 comprises a bone portion 508 and a cartilage layer 512. In some embodiments, the grafts 504 may be allografts that are harvested as one-piece components from a cartilage/bone joint location in a cadaver, and thus the cartilage layer 512 is advantageously affixed to the bone portion 508. It will be recognized by those skilled in the art that during implantation of the graft 504 into a recipient patient, damaged cartilage and underlying bone is removed from a joint to be treated, thereby forming an osteochondral bore having a diameter advantageously sized to receive the graft 504. The graft 504 is then inserted into the bore such that the surface of the cartilage layer 512 is aligned with the surrounding cartilage, thus encouraging healing and incorporation of the graft 504 into the patient's joint. As such, the cartilage layer 512 preferably comprises a thickness which closely matches the thickness of the existing cartilage in the patient's joint. In some embodiments, the cartilage layer 512 comprises a thickness which depends upon the location in the cadaver from where the graft 504 is harvested. In some embodiments, the cartilage layer 512 is roughly 2 mm in thickness.

It is contemplated that the grafts 504 may be comprised of any of various synthetic implantable materials, without limitation. For example, the cartilage layer 512 may be comprised of any of various biostable polyurethanes, such as polycarbonate-urethane (PCU) or thermoplastic silicone-polycarbonate-urethane (TSPCU). As will be appreciated, PCU materials generally possess durability, elasticity, fatigue and wear resistance, as well as compliance and tolerance in the body during healing, and thus are suitable for long-term implantation. The modulus of elasticity of implantable polyurethanes is known to be similar to that of articular cartilage, and thus it is contemplated that PCU materials may be suitable for use as the cartilage layer 512. Further, in some embodiments, the cartilage layer 512 may be comprised of polyvinyl alcohol (PVA), a synthetic polymer derived from polyvinyl acetate through partial or full hydroxylation. It is contemplated that PVA is suitable for use as artificial cartilage and meniscus due to the low protein adsorption characteristics, biocompatibility, high water solubility, and chemical resistance of PVA.

Moreover, in some embodiments, the grafts 504 may be of a xenograft variety, wherein either or both of the bone portion 508 and the cartilage layer 512 may be harvested from a donor species and then grafted into the patient's joint, as described herein. For example, in some embodiments, the grafts 504 may be comprised of collagen, bone, and/or cartilage that is bovine or porcine in origin. The grafts 504 may be harvested as one-piece components from suitable cartilage/bone joint locations in a donor animal, such that the cartilage layer 512 is affixed to the bone portion 508 and is suitable for implantation in the joint to be treated.

It is envisioned that the grafts 504 are not to be limited to xenografts or allografts, nor limited to the above-mentioned synthetic materials. Rather, it is contemplated that either or both of the bone portion 508 and the cartilage layer 512 may be comprised of any material(s) that may be found to be suitable for implantation in the joint to be treated, without limitation. For example, in some embodiments, the bone portion 508 comprising any of the one or more grafts 504 may comprise a cylindrical or tapered first implant material such as a monophasic allograft or autograft suitable for treating an osteochondral/subchondral defect. The cartilage layer 512 may comprise a disc-shaped second implant material that is configured in the form of a membrane to be placed on top of the bone portion 508, once implanted in a subchondral hole, so as to form an implant comprising a two-piece material. It is contemplated that the second implant material may comprise any of one or more of collagen, human allograft membrane, human allograft membrane, animal xenograft membrane, bioglass, PGA, PLLA, Calcium phosphate, silicone, peek, polyethylene, titanium, cobalt chrome, and the like, without limitation. It is further contemplated that the first material may comprise any of one or more of collagen, animal xenograft, human allograft, human autograft, silicone, bioglass, peek, polyethylene, titanium, cobalt chrome, and the like, without limitation.

As shown in FIGS. 26 and 27, the bone portion 508 comprises a multiplicity of surface features configured so as to promote the recipient patient's bone tissue to grow into the bone portion 508, thereby accelerating incorporation of the graft 504 into the patient's bone. In the embodiments illustrated in FIGS. 26 and 27, the surface features comprise holes 516 and longitudinal grooves 520. In some embodiments, the holes 516 may be relatively shallow so as to form dimples on the sides of the bone portion 508. In some embodiments, the holes 516 may be relatively deep, or extend all the way across the diameter of the bone portion 508. Further, various diameter sizes of the holes 516 may be implemented depending upon the size of the grafts 504 and the locations within the patient's body for which the grafts 504 are intended to be implanted.

Similarly, the longitudinal grooves 520 may be implemented with a variety of widths, lengths, and depths within the bone portion 508. Moreover, any number of the longitudinal grooves 520 may be formed into the bone portion 508 and distributed around the circumference of the graft 504. As will be appreciated, the specific number and dimensions of the longitudinal grooves 520 may be implemented based on the sizes of the grafts 504 and the locations within the patient's body where the grafts 504 are to be implanted. Further, the longitudinal grooves 520 may be implemented with a wide variety of cross-sectional shapes. In some embodiments, the longitudinal grooves 520 comprise a hemispherical cross-sectional shape. In some embodiments, the longitudinal grooves 520 comprise a rectangular cross-sectional shape. In some embodiments, the longitudinal grooves 520 comprise a triangular, or wedge, cross-sectional shape. Moreover, the longitudinal grooves 520 incorporated into an individual graft 504 are not limited to possessing the same cross-sectional shape, but rather various cross-sectional shapes may be applied to the longitudinal grooves 520 formed on each individual graft 504. It should be understood, therefore, that individual grafts 504 need not be limited to one type of surface feature, but rather different types of surface features may be mixed and incorporated into each of the grafts 504. Further, surface features other than holes and longitudinal grooves, as may become apparent to those skilled in the art, may be incorporated into the grafts 504 without going beyond the scope of the present disclosure.

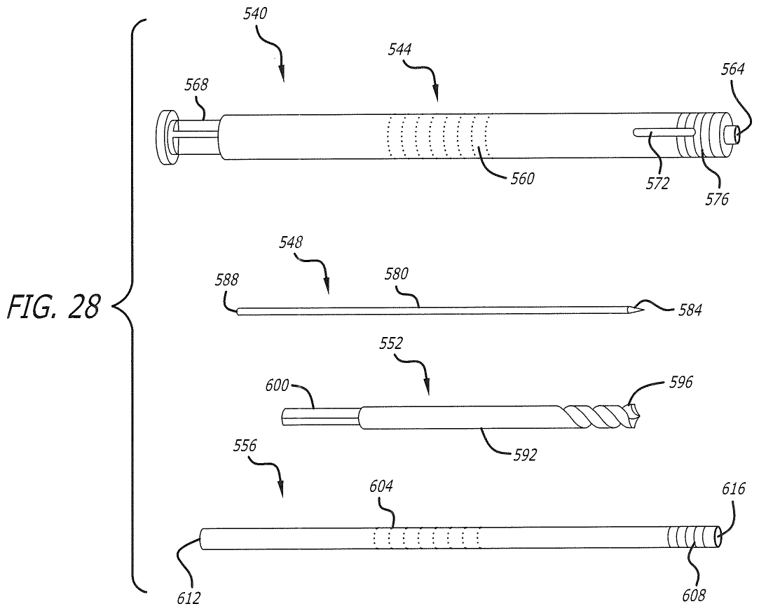

FIG. 28 illustrates a perspective view of an exemplary embodiment of an instrument kit 540 configured for implanting the grafts 504 into bone joints of a patient, as described herein. In the embodiment illustrated in FIG. 28, the instrument kit 540 comprises a graft inserter 544, a guidewire 548, a reamer 552, and a size gauge 556. In some embodiments, the instrument kit 540 may further comprise a tamp, similar to the insertion tamp 480 (see FIG. 22). As will be appreciated, the sterile plug system 500 comprises instruments necessary to perform cartilage graft implant surgeries. The sizes of the instruments comprising the kit 540 will depend upon the size of the particular graft 504 to be implanted into the patient. It is envisioned, therefore, that a surgeon may select one or more of the grafts 504 and a correspondingly sized embodiment of the instrument kit 540 based on the location and size of the bone joint to be treated.

Referring still to FIG. 28, the graft inserter 544 comprises a generally elongate member 560 having a distal graft retainer 564 and a proximal applicator 568. The proximal applicator 568 is in mechanical communication with the distal graft retainer 564 by way of an interior channel of the elongate member 560. The distal graft retainer 564 comprises an opening configured to receive and advantageously hold the graft 504 while the graft inserter 544 is used to direct the graft 504 to an implant location within the patient. As will be appreciated, the implant location generally is a surgically performed osteochondral bore formed to remove damaged articular cartilage and a portion of the underlying bone tissue so as to accommodate implantation of the graft 504. As such, the osteochondral bore has a diameter and a depth suitable to receive the graft 504, such that the cartilage layer 512 aligns with surrounding healthy cartilage in the bone joint. Once the graft 504 is suitably positioned at the implant location, the proximal applicator 568 may be used to push the graft 504 out of the distal graft retainer 564 and into the osteochondral bore.

A viewport 572 facilitates directly observing the position of the graft 504 within the distal graft retainer 564. Further, the viewport 572 facilitates observing the length of the graft by way of a graft length indicator 576. The graft length indicator 576 comprises a series of ring lines positioned adjacent to the viewport 572 with a sequentially increasing distance from the distal graft retainer 564. As will be appreciated, when the graft 504 is fully received into the distal graft retainer 564, the position of the top of the cartilage layer 512 relative to the graft length indicator 576 provides a visual indication of the total length of the graft 504. Thus, the viewport 572 and the graft length indicator 576 advantageously enables the surgeon to verify that a correctly sized graft 504 has been selected for surgery.

As illustrated in FIG. 28, the guidewire 548 comprises an elongate shaft 580 having a distal pointed tip 584 and a proximal blunt end 588. The guidewire 548 is configured to be inserted into confined spaces within bone joints and serves to direct a subsequent insertion of the reamer 552 and the size gauge 556 to the implant location within the bone joint. In some embodiments, the guidewire 548 is comprised of a surgical stainless steel, such as austenitic 316 stainless steel, martensitic 440 stainless steel, martensitic 420 stainless steel, and the like. It will be appreciated that the distal pointed tip 584 facilitates advancing the guidewire 548 through obstructive tissues and structures, and the proximal blunt end 588 facilitates manipulating the guidewire 548 by hand, or by way of an appropriate tool.

The reamer 552 comprises a rigid elongate shaft 592 having a distal cutting end 596 and a proximal shank 600. The distal cutting end 596 comprises a cutting edge suitable for rotatably clearing an osteochondral bore, thereby removing damaged articular cartilage and an underlying bone portion from the bone joint being treated. In some embodiments, the distal cutting end 596 comprises a spiral cutting edge, although other suitable cutting edge configurations will be apparent. The proximal shank 600 is configured to be grasped by a chuck of a surgical drill, or other equivalent rotary tool. Further, in some embodiments the reamer 552 may comprise a central, lengthwise hole whereby the reamer may be mounted onto the guidewire 548 so as to direct the distal cutting end 596 to the implant location within the bone joint.

With continuing reference to FIG. 28, the size gauge 556 comprises a generally elongate member 604 having a depth indicator 608 and a proximal handle portion 612. The size gauge 556 further comprises a central, lengthwise hole 616 having a diameter suitable to receive the guidewire 548. The central hole 616 facilitates mounting the size gauge onto the guidewire 548 so as to direct the depth indicator 608 to the osteochondral bore formed within the bone joint. The depth indicator 608 comprises a series of ring lines positioned on the elongate member with a sequentially increasing distance from a distal end of the size gauge 556. As will be appreciated, upon inserting the depth indicator 608 fully into the osteochondral bore, the ring lines provide the surgeon with a direct observation of the depth of the bore. It should be understood that the depth indicator 608 generally correlates with the graft length indicator 576 of the graft inserter 544 so as to ensure that the osteochondral bore is drilled to a depth suitable to accommodate the graft 504, such that the cartilage layer 512 aligns with the surrounding cartilage within the bone joint.

It is to be understood that the instrument kit 540 is not to be limited to the specific instruments shown in FIG. 28. For example, in some embodiments, any one or more of the size gauges 280, 296, 320 may be included in the instrument kit 540. In some embodiments, the guidewire 344 may be included in the instrument kit 540, lieu of the guidewire 548. Further, in some embodiments, the cannulated reamer 440 may be included in the instrument kit 540, in lieu of the reamer 552. In some embodiments, the cartilage punch 380, the cannulated obturator 400, and the cannulated reamer 440 may be included in the instrument kit 540, in lieu of the reamer 552. In some embodiments, the insertion tamp 480 may be included in the instrument kit 540, without limitation. Furthermore, in some embodiments, the instrument kit 540 may include any one or more of the size gauges 280, 296, 320, the guidewire 344, the cartilage punch 380, the cannulated obturator 400, the cannulated reamer 440, and the insertion tamp 480, without limitation.

Moreover, the sterile implant system 180 is not to be limited to the specific instruments shown in FIGS. 6-7B, nor is the system 180 to be limited to the number of instruments shown in FIGS. 6-7B. For example, in some embodiments, any one or more of the size gauges 280, 296, 320 may be included in the sterile implant system 180, in lieu of the size gauge 188. In some embodiments, the guidewire 344 may be included in the sterile implant system 180, lieu of the guidewire 192. Further, in some embodiments, the cannulated reamer 440 may be included in the sterile implant system 180, in lieu of the cannulated reamer 196. In some embodiments, the cartilage punch 380, the cannulated obturator 400, and the cannulated reamer 440 may be included in the sterile implant system 180, in lieu of the cannulated reamer 196. In some embodiments, the insertion tamp 480 may be included in the sterile implant system 180, without limitation. Furthermore, in some embodiments, the sterile implant system 180 may include any one or more of the size gauges 280, 296, 320, the guidewire 344, the cartilage punch 380, the cannulated obturator 400, the cannulated reamer 440, and the insertion tamp 480, without limitation.

In general, it is contemplated that the sterile implant system 180 of FIG. 6 is to be suitably sterilized for surgeries and packaged into sterilized containers. In some embodiments, the size gauge 188 is packaged in a first sterile container, while the guidewire 192, the cannulated reamer 196, the punch 260, and a graft inserter, if included, are packaged in a second sterile container, and the tapered implant 184 is packaged in a third sterile container. In some embodiments, the first, second, and third sterile containers may then be bundled together into a single, exterior container, thereby forming a convenient surgery-specific cartilage repair package. In some embodiments, however, the second and third sterile containers may be bundled together into a single, exterior container while the first sterile container is packaged into a dedicated exterior container.

Similarly, the instrument kit 540 of FIG. 28 is to be suitably sterilized for surgeries and packaged into sterilized containers. The size gauge 556 may be packaged in a first sterile container while the graft inserter 544, the guidewire 548, and the reamer 552 are packaged in a second sterile container, and the graft 504 is packaged in a third sterile container. The first, second, and third sterile containers may then be bundled together into a single, exterior container, thereby forming a convenient surgery-specific cartilage graft package. It is envisioned that other packaging techniques will be apparent to those skilled in the art without deviating from the spirit and scope of the present disclosure.

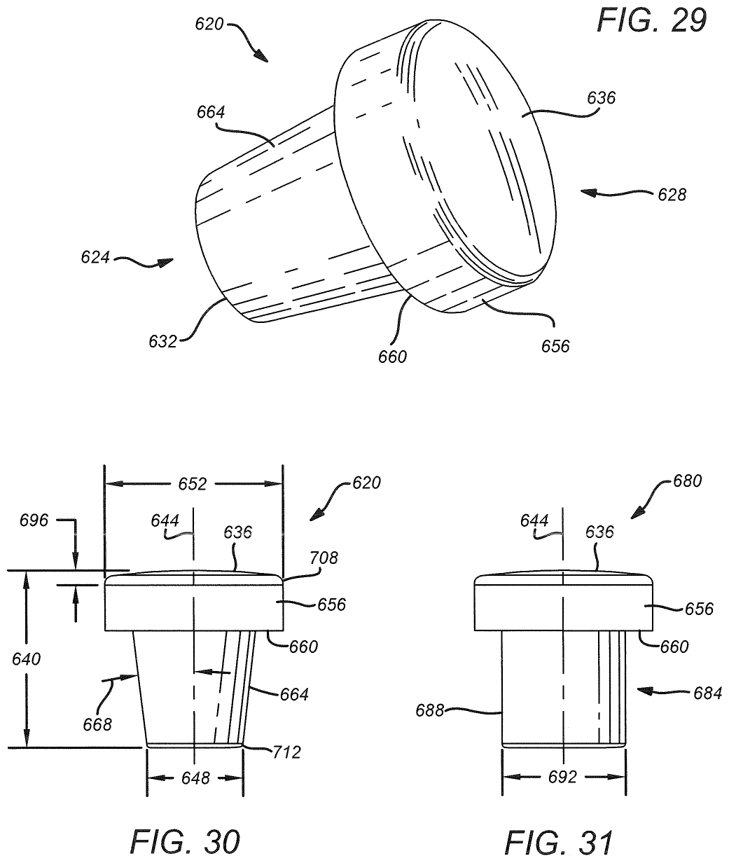

FIG. 29 illustrates an exemplary embodiment of a tapered osteochondral implant 620 for treating osteochondral/subchondral defects in accordance with the present disclosure. The implant 620 includes a lower portion 624 and an upper portion 628. The implant 620 is configured to be press-fit into an osteochondral hole bored at a patient's defect area. The lower portion 624 includes a bottom surface 632 configured to be implanted into the osteochondral hole drilled into the patient's bone. The upper portion 628 includes a top surface 636 that includes a shape that approximates an osteochondral surface to be replaced. The implant 620 may comprise any synthetic or natural homogenous material suitable for implantation into bone, including any one or more of collagen, animal xenograft, human allograft, human autograft, silicone, bioglass, collagen, peek, polyethylene, titanium, cobalt chrome, and the like. In some embodiments, the implant 620 is comprised of a material exhibiting a hardness of at least 30 durometer.

It is contemplated that the implant 620 may be implemented with a range of dimensions that facilitate using the implant 620 to treat osteochondral or subchondral defects in various bone joint locations in the human body, such as by way of non-limiting example, a femoral condyle, a humeral head, a talus, the trapezium of the hand, the capitellum of the elbow, as well as any of the metatarsal and phalangeal joints of the hand or foot. As shown in FIG. 30, for example, the implant 620 possesses a height 640 along a longitudinal axis 644 of the implant and a bottom diameter 648 centered on the longitudinal axis 644. The upper portion 628 includes a top diameter 652 centered on the longitudinal axis 644. The height 640 generally extends from the bottom surface 632 to the highest region of the top surface 636, such as the region of the top surface 636 around the longitudinal axis 644. In some embodiments, the height 640 may range between about 13 mm and 16 mm. It is contemplated, however, that the height 640 may be varied according to the bone joint to be treated, and thus the implant 620 may be implemented with a wide variety of heights 640, without limitation.

The upper portion 628 includes a cylindrical sidewall 656 that comprises an untapered, or straight cylindrical shape extending from a periphery of the top surface 636 to a flat undersurface 660 of the upper portion 628, as best shown in FIG. 30. Thus, the cylindrical sidewall 656 shares the same diameter as the top diameter 652 of the top surface 636. In some embodiments, the top diameter 652 may range between about 11 mm and 13 mm. It is contemplated, however, that the top diameter 652 may be varied according to the bone joint to be treated, and thus the implant 620 may be implemented with a wide variety of diameters, including tapered diameters, without limitation.

The lower portion 624 includes a cylindrical sidewall 664 that includes a taper that causes a diameter of the sidewall 664 to decrease from an initial diameter at the undersurface 660 to the bottom diameter 648 of the bottom surface 632. As shown in FIG. 30, the taper of the sidewall 664 may be expressed in terms of a taper half-angle 668 taken with respect to the longitudinal axis 644. The taper of the sidewall 664 is configured to prevent the implant 620 from subsiding into the osteochondral hole drilled in bone. In one embodiment, for example, the taper half-angle 668 is substantially 6.0 degrees.

It should be borne in mind that the taper half-angle 668 may be any angle that is found to prevent subsidence of the implant 620, including an angle of zero degrees, without limitation. For example, FIG. 31 illustrates an exemplary embodiment of an osteochondral implant 680 that is substantially identical to the implant 620, shown in FIG. 30, with the exception that the implant 680 includes a lower portion 684 having an untapered, straight cylindrical sidewall 688. As such, the diameter of the sidewall 688 generally is uniform from the undersurface 660 to a bottom diameter 692. Further, it will be recognized that the uniform diameter of the sidewall 688 gives rise to a larger bottom diameter 692 of the implant 680 than the bottom diameter 648 of the implant 620.

With reference again to FIG. 30, in some embodiments the overall size of the implant 620 may be identified based on the bottom diameter 648 without a specific reference to the included taper half-angle 668 of the implant 620. In such embodiments, a practitioner may select the implant 620 based on a size of the osteochondral hole to be drilled into the patient's bone. As with other dimensions of the implant 620 discussed hereinabove, however, the bottom diameter 648 may be varied according to the bone joint to be treated. In one embodiment, the bottom diameter 648 ranges between roughly 5 mm and about 10 mm. As will be appreciated, therefore, the implant 620 may be implemented with a wide variety of bottom diameters 648, without limitation.

Moreover, in some embodiments the overall size of the implant 620 may be identified based on the top diameter 652 of the top surface 636, and thus the size of the implant 620 may be selected based on the area of the joint defect to be treated. For example, as mentioned hereinabove, the top diameter 652 may range between about 11 mm and 13 mm. It is contemplated that in such embodiments, the specific sizes of the bottom diameter 648 and the taper half-angle 668 may be incorporated into the implant 620 in accordance with the diameter of the top surface 636, and thus the sizes of the bottom diameter 648 and the taper half-angle 668 need not be specifically called out. For example, in some embodiments, any one or more of the height 640, the taper half-angle 668, and the bottom diameter 648 of the implant 620 may be configured to correlate with the top diameter 652 of the top surface 636, without limitation.

As further shown in FIG. 30, the top surface 636 includes a positive curvature height 696 that imparts a convex curvature to the implant 620. The positive curvature height 692 may be used to dispose the top surface 636 of the implant 620 slightly above surrounding cartilage tissue of the bone to be treated. In general, however, the top surface 636 includes a shape configured to approximate the osteochondral or subchondral surface to be replaced. For example, in some embodiments, the shape of the top surface 636 includes a curvature that approximates the curvature of the osteochondral surface to be replaced. As such, in some embodiments, the top surface 636 includes a concave, curvature that corresponds to a negative curvature height 696 of the implant 620. It is contemplated that an embodiment of the implant 620 that includes a negative curvature height 696 may be advantageously configured for treating cartilage defects in the 1^{st} proximal phalangeal bone, while an embodiment of the implant 620 that includes a positive curvature height 696 may be configured for treating cartilage defects in the 1^{st} metatarsal bone. For subchondral implants, the top surface 636 may have a flat curvature, without limitation, as the implant generally is disposed below the surrounding articular surface and thus does not need to approximate the shape of articular surface.

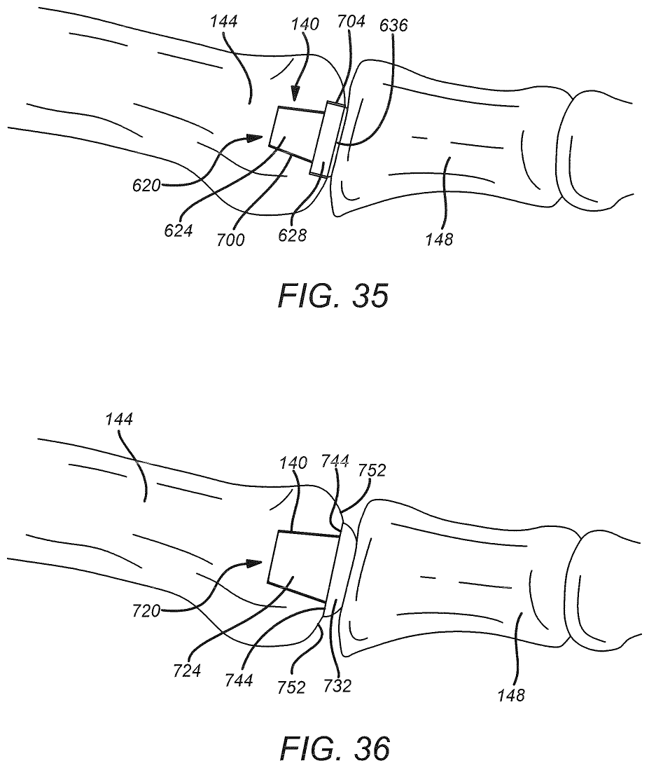

FIG. 35 illustrates an exemplary use environment wherein the tapered osteochondral implant 620 is implanted into an osteochondral hole 140 drilled in a 1^{st} metatarsal bone 144. As will be recognized, the top surface 636 of the implant 620 is disposed slightly above the surrounding cartilage tissue of the 1^{st} metatarsal bone 144 and in contact with an adjacent 1st proximal phalangeal bone 148. In general, the top surface 636 includes a shape configured to approximate the osteochondral surface to be replaced. In some embodiments, such as the illustrated embodiment of FIG. 35, the shape of the top surface 636 includes a convex curvature (see FIG. 30) that approximates the curvature of the osteochondral surface to be replaced. In embodiments of the top surface 636 including a convex curvature, the implant 620 includes a positive curvature height 696 as shown in FIG. 30. As mentioned above, the top surface 636 may, in some embodiments, include a concave curvature that corresponds to a negative curvature height 696 of the implant 620. It is contemplated that an embodiment of the implant 620 including a negative curvature height 696 is advantageously configured for treating cartilage defects in the 1^{st} proximal phalangeal bone 148.

As shown in FIG. 35, the osteochondral hole 140 may include a lower, tapered portion 700 and an upper, untapered portion 704. It is contemplated that the tapered portion 700 generally includes a tapered diameter suitable for contacting the lower portion 624 of the implant 620. Similarly, the untapered portion 704 is configured to receive the sidewall 656 of the upper portion 628 such that the sidewall 656 contacts the surrounding bone within the osteochondral hole 140. As will be appreciated, a suitable cannulated reamer may be advantageously adapted to drill the tapered and untapered portions 700, 704 comprising the osteochondral hole 140. For example, the cannulated reamer 196, shown in FIG. 6, may be configured to include a first portion to drill the tapered portion 700 and a second, stepped portion configured to drill the untapered portion 704.

As further shown in FIG. 35, the implant 620 includes a height 640 (see FIG. 30) that places the bottom surface 632 in contact with a bottom of the osteochondral hole 140 and elevates the top surface 636 slightly above the surrounding cartilage tissue of the 1^{st} metatarsal bone 144. The taper half-angle 668 advantageously prevents subsidence of the implant 620 into the osteochondral hole 140, even in the event that the bone below the bottom surface 632 subsides. As best illustrated in FIG. 30, the implant 620 may include a rounded periphery 708 that joins the top surface 636 and the cylindrical sidewall 656. The rounded periphery 708 comprises a transition surface between the top surface 636 and the sidewall 656 that provides a smooth contact surface to surrounding tissues. Further, the implant 620 includes a rounded periphery 712 that joins the cylindrical sidewall 664 and the bottom surface 632. As will be appreciated, the rounded periphery 712 provides a smooth transition surface between the sidewall 664 and the bottom surface 632 that prevents damage to the interior sidewalls of the osteochondral hole 140 during insertion of the implant 620 therein.

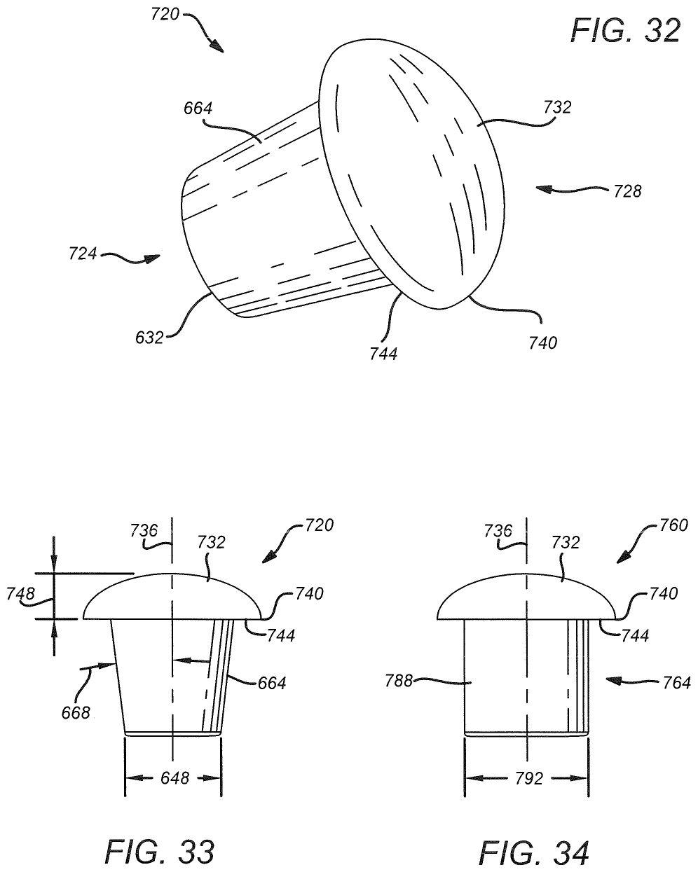

Turning, now, to FIG. 32, an exemplary embodiment of a tapered osteochondral implant 720 for treating osteochondral/subchondral defects is shown. The implant 720 may comprise any synthetic or natural homogenous material suitable for implantation into bone, including any one or more of collagen, animal xenograft, human allograft, human autograft, silicone, bioglass, collagen, peek, polyethylene, titanium, cobalt chrome, and the like. In some embodiments, the implant 720 is comprised of a material exhibiting a hardness of at least 30 durometer.

The implant 720 includes a lower portion 724 and an upper portion 728. The lower portion 724 is substantially identical to the lower portion 624 of the implant 620 shown in FIG. 29, and thus the lower portion 724 includes a bottom surface 632 and a sidewall 664 that extends to the upper portion 728. The upper portion 728 includes a rounded top surface 732 that extends from a longitudinal axis 736 (see FIG. 33) to a periphery 740 of the upper portion 728. As best shown in FIG. 33, a flat undersurface 744 extends inward from the periphery 740 to the tapered sidewall 664 of the lower portion 624.

As will be appreciated, the top surface 732 includes a positive curvature height 748 (see FIG. 33) that imparts a convex curvature to the implant 720. The positive curvature height 748 may be used to dispose the top portion 728 of the implant 720 above surrounding cartilage tissue of the bone to be treated. For example, when the implant 720 is pressed into an osteochondral hole 140 drilled at a defect area of a patient's 1^{st} metatarsal bone 144, as shown in FIG. 36, the lower portion 724 may be implanted into the osteochondral hole 140 while the undersurface 744 contacts an exterior surface 752 of the cartilage tissue surrounding the osteochondral hole 140. As shown in FIG. 36, the top surface 732 of the implant 720 contacts an adjacent 1^{st} proximal phalangeal bone 148. It is contemplated, however, that in some embodiments of the implant 720 at least a portion of the top surface 732 may include a negative curvature height that is advantageously configured for treating cartilage defects in the 1^{st} proximal phalangeal bone, without limitation.

Turning again to FIG. 33, the lower portion 724 includes a cylindrical sidewall 664 that includes a taper that causes a diameter of the sidewall 664 to decrease from an initial diameter at the undersurface 744 to a bottom diameter 648 of the bottom surface 632. As described herein, the taper of the sidewall 664 may be expressed in terms of a taper half-angle 668 taken with respect to the longitudinal axis 736. The taper of the sidewall 664 is configured to prevent the implant 720 from subsiding into the osteochondral hole drilled in bone. In one embodiment, for example, the taper half-angle 668 is substantially 6.0 degrees.

As mentioned hereinabove, the taper half-angle 668 may be any angle that is found to prevent subsidence of the implant 720, including an angle of zero degrees, without limitation. For example, FIG. 34 illustrates an exemplary embodiment of an osteochondral implant 760 that is substantially identical to the implant 720, shown in FIG. 33, with the exception that the implant 760 includes a lower portion 764 having an untapered, straight cylindrical sidewall 788. As such, the diameter of the sidewall 788 generally is uniform from the undersurface 744 to a bottom diameter 792. Further, the uniform diameter of the sidewall 788 gives rise to a larger bottom diameter 792 of the implant 760 than the bottom diameter 648 of the implant 720.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. An osteochondral implant for treating osteochondral/subchondral defects, the implant comprising:
a lower portion including a bottom surface for being pressed into an osteochondral hole drilled at a defect area; and
an upper portion including a top surface for replacing an osteochondral surface.

2. The implant of claim 1, wherein at least one of the lower portion and the upper portion comprises any synthetic or natural homogenous material suitable for implantation into bone, including any one or more of collagen, human allograft or human autograft, or animal xenograft, silicone, bioglass, collagen, peek, polyethylene, titanium, or cobalt chrome.

3. The implant of claim 1, wherein at least one of the lower portion and the upper portion comprises a material exhibiting a hardness of at least 30 durometer.

4. The implant of claim 1, wherein the upper portion includes a cylindrical sidewall that extends from a periphery of the top surface to a flat undersurface.

5. The implant of claim 4, wherein the lower portion includes a cylindrical sidewall having a diameter that is substantially uniform from the undersurface to the bottom surface.

6. The implant of claim 4, wherein the lower portion includes a cylindrical sidewall having a diameter that decreases from an initial diameter at the undersurface to a bottom diameter of the bottom surface.

7. The implant of claim 6, wherein the decreasing diameter of the cylindrical sidewall is configured to prevent the implant from subsiding into the osteochondral hole.

8. The implant of claim 1, wherein the top surface includes a positive curvature height that imparts a convex curvature to the upper portion.

9. The implant of claim 8, wherein the positive curvature height is configured to dispose the top surface slightly above cartilage tissue surrounding the defect area to be treated.

10. The implant of claim 8, wherein the top surface includes a shape configured to approximate the osteochondral or subchondral surface to be replaced.

11. The implant of claim 1, wherein the top surface includes a positive curvature that extends to a periphery that joins an undersurface of the upper portion.

12. The implant of claim 11, wherein the undersurface extends inward from the periphery to a cylindrical sidewall comprising the lower portion.

13. The implant of claim 12, wherein the undersurface is configured to contact an exterior surface of the cartilage tissue surrounding the defect area to be treated.

14. The implant of claim 1, wherein the lower portion is configured to be pressed into a subchondral hole such that the bottom surface contacts a bottom of the subchondral hole.

15. The implant of claim 1, wherein the upper portion includes a cylindrical sidewall configured to contact surrounding bone within a subchondral hole.

16. The implant of claim 1, wherein the lower portion comprises a first implant material including any of a homogenous synthetic material, a homogenous natural material, or a combination thereof.

17. The implant of claim 16, wherein the first implant material comprises any one or more of collagen, animal xenograft, human allograft, human autograft, silicone, bioglass, peek, polyethylene, titanium, or cobalt chrome.

18. The implant of claim 16, wherein the upper portion comprises a second implant material configured to be placed on top of the first implant material to form a two-piece construct of the implant.

19. The implant of claim 18, wherein the second implant material comprises any one or more of collagen, animal xenograft, human allograft, human autograft, bioglass, PGA, PLLA, Calcium phosphate, silicone, peek, polyethylene, titanium, or cobalt chrome.

20. A method for treating an osteochondral/subchondral defect, the method comprising:
drilling a subchondral hole at a defect area of a joint;
pressing a lower portion comprising a two-piece implant into the subchondral hole; and
placing an upper portion comprising the two-piece implant on top of the lower portion.

21. The method of claim 20, wherein pressing includes using a first implant material comprising the lower portion that includes any of a homogenous synthetic material, a homogenous natural material, or a combination thereof.

22. The method of claim 21, wherein the first implant material comprises any one or more of collagen, animal xenograft, human allograft, human autograft, silicone, bioglass, peek, polyethylene, titanium, or cobalt chrome.

23. The method of claim 20, wherein placing includes selecting a second implant material comprising the upper portion that includes any one or more of collagen, human allograft membrane, human autograft membrane, animal xenograft membrane, bioglass, PGA, PLLA, Calcium phosphate, silicone, peek, polyethylene, titanium, or cobalt chrome.
